SEQUENCE LISTING (56 sequences)

SEQ ID NO: 1 - protein, 8 aa, Artificial Sequence
  CDR1 of msVH22.101 and hVH22.101(HC)x
GYTFTNYG

SEQ ID NO: 2 - protein, 8 aa, Artificial Sequence
  CDR2 of msVH22.101 and hVH22.101(HC)x
INTYSGEA

SEQ ID NO: 3 - protein, 15 aa, Artificial Sequence
  CDR3 of msVH22.101 and hVH22.101(HC)x
LRGYTYQSFDEGGDY

SEQ ID NO: 4 - protein, 3 aa, Artificial Sequence
  CDR2 of msVL22.101 and hVL22.101(LC)y
LVS

SEQ ID NO: 5 - protein, 9 aa, Artificial Sequence
  CDR3 of msVL22.101 and hVL22.101(LC)y
WQGTHFPYT

SEQ ID NO: 6 - protein, 11 aa, Artificial Sequence
  CDR1 of hVL22.101LC17
QSLLDTDGKTY

SEQ ID NO: 7 - protein, 11 aa, Artificial Sequence
  CDR1 of hVL22.101LC21
QSLLDSDAKTY

SEQ ID NO: 8 - protein, 11 aa, Artificial Sequence
  CDR1 of hVL22.101LC27
QSLLDTDAKTY

SEQ ID NO: 9 - protein, 11 aa, Artificial Sequence
  CDR1 of hVL22.101LC41
QSLLDADGKTY

SEQ ID NO: 10 - protein, 11 aa, Artificial Sequence
  CDR1 of hVL22.101LC42
QSLLDNDGKTY

SEQ ID NO: 11 - protein, 122 aa, Artificial Sequence
  hVH22.101f
RIQLVQSGAEVKKPGASVKVSCKASGYTFTNYGMHWVRQAPGQGLEWMGWINTYSGEATYAQKFQGRVTMTRDTSISTAYMELSRLRSDDTAVYYCLRGYTYQSFDEGGDYWGQGTLVTVSS

SEQ ID NO: 12 - protein, 122 aa, Artificial Sequence
  hVH22.101HC9
RIQLVQSGAEVKKPGASVKVSCKASGYTFTNYGMHWVRQAPGQGLEWMGWINTYSGEATYVDDFQGRVTMTRDTSISTAYMELSRLRSDDTAVYYCLRGYTYQSFDEGGDYWGQGTLVTVSS

SEQ ID NO: 13 - protein, 112 aa, Artificial Sequence
  hVL22.101LC17
DVVMTQSPLSLPVTLGQPASISCRSSQSLLDTDGKTYLNWFQQRPGQSPRRLIYLVSKLDSGVPDRFSGSGSGTDFTLKISRVEAEDVGVYYCWQGTHFPYTFGQGTKLEIK

SEQ ID NO: 14 - protein, 112 aa, Artificial Sequence
  hVL22.101LC21
DVVMTQSPLSLPVTLGQPASISCRSSQSLLDSDAKTYLNWFQQRPGQSPRRLIYLVSKLDSGVPDRFSGSGSGTDFTLKISRVEAEDVGVYYCWQGTHFPYTFGQGTKLEIK

SEQ ID NO: 15 - protein, 112 aa, Artificial Sequence
  hVL22.101LC27
DVVMTQSPLSLPVTLGQPASISCRSSQSLLDTDAKTYLNWFQQRPGQSPRRLIYLVSKLDSGVPDRFSGSGSGTDFTLKISRVEAEDVGVYYCWQGTHFPYTFGQGTKLEIK

SEQ ID NO: 16 - protein, 112 aa, Artificial Sequence
  hVL22.101LC41
DVVMTQSPLSLPVTLGQPASISCRSSQSLLDADGKTYLNWFQQRPGQSPRRLIYLVSKLDSGVPDRFSGSGSGTDFTLKISRVEAEDVGVYYCWQGTHFPYTFGQGTKLEIK

SEQ ID NO: 17 - protein, 112 aa, Artificial Sequence
  hVL22.101LC42
DVVMTQSPLSLPVTLGQPASISCRSSQSLLDNDGKTYLNWFQQRPGQSPRRLIYLVSKLDSGVPDRFSGSGSGTDFTLKISRVEAEDVGVYYCWQGTHFPYTFGQGTKLEIK

SEQ ID NO: 18 - protein, 12 aa, Artificial Sequence
  synthetic peptide
  MISC_FEATURE (3)..(3): X is citrulline
SGXGKQGGKARA

SEQ ID NO: 19 - protein, 23 aa, Artificial Sequence
  synthetic peptide
  MISC_FEATURE (3)..(3): X is citrulline
SGXGKGGKGLGKGGAKRHRKVLR

SEQ ID NO: 20 - protein, 12 aa, Artificial Sequence
  synthetic peptide
  MISC_FEATURE (3)..(3): X is citrulline
SGXGKGGKGLGK

SEQ ID NO: 21 - protein, 12 aa, Artificial Sequence
  synthetic peptide
  MISC_FEATURE (6)..(6): X is citrulline
QFPVGXVHRLLR

SEQ ID NO: 22 - protein, 12 aa, Artificial Sequence
  synthetic peptide
  MISC_FEATURE (6)..(6): X is citrulline
VHRLLXKGNYSE

SEQ ID NO: 23 - protein, 329 aa, Artificial Sequence
  Human heavy chain constant domain of IgG1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 24 - protein, 107 aa, Artificial Sequence [sequence omitted]

SEQ ID NO: 25 - protein, 122 aa, Artificial Sequence
  msVH22.101
RIQLVQSGPELKKPGEAVKISCKASGYTFTNYGMHWMKQTPGKDFRWMGWINTYSGEATYVDDFKGRFAFSLGTSASTAYLQINNLKNDDTATYFCLRGYTYQSFDEGGDYWGQGTALTVSS

SEQ ID NO: 26 - protein, 122 aa, Artificial Sequence
  hVH22.101j
QVQLVQSGAEVKKPGASVKVSCKASGYTFTNYGMHWVRQAPGQGLEWMGWINTYSGEATYAQKFQGRVTMTRDTSISTAYMELSRLRSDDTAVYYCLRGYTYQSFDEGGDYWGQGTLVTVSS

SEQ ID NO: 27 - protein, 122 aa, Artificial Sequence
  hVH22.101HC7
QVQLVQSGAEVKKPGSSVKVSCKASGYTFTNYGMHWVRQAPGQGLEWMGWINTYSGEATYAQKFQGRVTITADESTSTAYMELSSLRSEDTAVYYCLRGYTYQSFDEGGDYWGQGTLVTVSS

SEQ ID NO: 28 - protein, 122 aa, Artificial Sequence
  hVH22.101HC8
QVQLVQSGAEVKKPGSSVKVSCKASGYTFTNYGMHWVRQAPGQGLEWMGWINTYSGEATYVDDFQGRVTITADESTSTAYMELSSLRSEDTAVYYCLRGYTYQSFDEGGDYWGQGTLVTVSS

SEQ ID NO: 29 - protein, 122 aa, Artificial Sequence
  hVH22.101HC10
QVQLVQSGAEVKKPGASVKVSCKASGYTFTNYGMHWVRQAPGQGLEWMGWINTYSGEATYVDDFQGRVTMTRDTSISTAYMELSRLRSDDTAVYYCLRGYTYQSFDEGGDYWGQGTLVTVSS

SEQ ID NO: 30 - protein, 112 aa, Artificial Sequence
  msVL22.101
DVVMTQTPLTLSVTTGQPASISCKSSQSLLDSDGKTYLNWLFQRPGQSPKRLIYLVSKLDSGVPDRFTGSGSGTDFTLKISRVEAEDLGIYYCWQGTHFPYTFGGGTNLEIK

SEQ ID NO: 31 - protein, 112 aa, Artificial Sequence
  hVL22.101e
DVVMTQSPLSLPVTLGQPASISCRSSQSLVDSDGKTYLNWFQQRPGQSPRRLIYLVSKLDSGVPDRFSGSGSGTDFTLKISRVEAEDVGVYYCWQGTHFPYTFGQGTKLEIK

SEQ ID NO: 32 - protein, 112 aa, Artificial Sequence
  hVL22.101g
DVVMTQSPLSLPVTLGQPASISCRSSQSLLDSDGKTYLNWFQQRPGQSPRRLIYLVSKLDSGVPDRFSGSGSGTDFTLKISRVEAEDVGVYYCWQGTHFPYTFGQGTKLEIK

SEQ ID NO: 33 - protein, 112 aa, Artificial Sequence
  hVL22.101h
DVVMTQSPLSLPVTLGQPASISCRSSQSLVASDGKTYLNWFQQRPGQSPRRLIYLVSKLDSGVPDRFSGSGSGTDFTLKISRVEAEDVGVYYCWQGTHFPYTFGQGTKLEIK

SEQ ID NO: 34 - protein, 112 aa, Artificial Sequence
  hVL22.101i
DVVMTQSPLSLPVTLGQPASISCRSSQSLVESDGKTYLNWFQQRPGQSPRRLIYLVSKLDSGVPDRFSGSGSGTDFTLKISRVEAEDVGVYYCWQGTHFPYTFGQGTKLEIK

SEQ ID NO: 35 - protein, 112 aa, Artificial Sequence
  hVL22.101j
DVVMTQSPLSLPVTLGQPASISCRSSQSLVSSDGKTYLNWFQQRPGQSPRRLIYLVSKLDSGVPDRFSGSGSGTDFTLKISRVEAEDVGVYYCWQGTHFPYTFGQGTKLEIK

SEQ ID NO: 36 - protein, 11 aa, Artificial Sequence
  CDR1 of msVL22.101 and hVL22.101g
QSLLDSDGKTY

SEQ ID NO: 37 - protein, 11 aa, Artificial Sequence
  CDR1 of hVL22.101e
QSLVDSDGKTY

SEQ ID NO: 38 - protein, 11 aa, Artificial Sequence
  CDR1 of hVL22.101h
QSLVASDGKTY

SEQ ID NO: 39 - protein, 11 aa, Artificial Sequence
  CDR1 of hVL22.101i
QSLVESDGKTY

SEQ ID NO: 40 - protein, 11 aa, Artificial Sequence
  CDR1 of hVL22.101j
QSLVSSDGKTY

SEQ ID NO: 41 - protein, 11 aa, Artificial Sequence
  CDR1 of hVL22.101LC16
QSLLESDGKTY

SEQ ID NO: 42 - protein, 11 aa, Artificial Sequence
  CDR1 of hVL22.101LC19
QSLLDSEGKTY

SEQ ID NO: 43 - protein, 11 aa, Artificial Sequence
  CDR1 of hVL22.101LC20
QSLLDSSGKTY

SEQ ID NO: 44 - protein, 11 aa, Artificial Sequence
  CDR1 of hVL22.101LC22
QSLLESEGKTY

SEQ ID NO: 45 - protein, 11 aa, Artificial Sequence
  CDR1 of hVL22.101LC23
QSLLESSGKTY

SEQ ID NO: 46 - protein, 11 aa, Artificial Sequence
  CDR1 of hVL22.101LC24
QSLLESDAKTY

SEQ ID NO: 47 - protein, 11 aa, Artificial Sequence
  CDR1 of hVL22.101LC25
QSLLDTEGKTY

SEQ ID NO: 48 - protein, 11 aa, Artificial Sequence
  CDR1 of hVL22.101LC26
QSLLDTSGKTY

SEQ ID NO: 49 - protein, 11 aa, Artificial Sequence
  CDR1 of hVL22.101LC37
QSLLDSAGKTY

SEQ ID NO: 50 - protein, 11 aa, Artificial Sequence
  CDR1 of hVL22.101LC38
QSLLESAGKTY

SEQ ID NO: 51 - protein, 11 aa, Artificial Sequence
  CDR1 of hVL22.101LC39
QSLLDAEGKTY

SEQ ID NO: 52 - protein, 11 aa, Artificial Sequence
  CDR1 of hVL22.101LC40
QSLLDNEGKTY

SEQ ID NO: 53 - protein, 17 aa, Artificial Sequence
  msFib  XG
  MISC_FEATURE (9)..(9): X is citrulline
EPTDSLDAXGHRPVDRR

SEQ ID NO: 54 - protein, 15 aa, Artificial Sequence
  msVim XS/XL
  MISC_FEATURE (4)..(4): X is citrulline
  MISC_FEATURE (9)..(9): X is citrulline
  MISC_FEATURE (11)..(11): X is citrulline
YVTXSSAVXLXSSVP

SEQ ID NO: 55 - protein, 7 aa, Artificial Sequence
  Region around CDR2 of msVL22.101 and hVL22.101(LC)y
LVSKLDS

SEQ ID NO: 56 - protein, 330 aa, Artificial Sequence [sequence omitted]

(19) United States
(12) Patent Application Publication
(10) Pub. No.: US 2024/0272145 A1
(43) Pub. Date: Aug. 15, 2024

(54) DIAGNOSTIC

(71) Applicant: CITRYLL B.V., OSS (NL)

(72) Inventors: RENATO GERARDUS SILVANO CHIRIVI, Oosterhout (NL); HELMUTH ADRIANUS MARIA VAN ES, OOSTERHOUT (CR); HELMUTH HENDRIKUS GERA VAN ES, Amsterdam (NL); MARINUS ADRIANUS MARIA VAN DER LINDEN, Sint-Oedenrode (NL); REZIE TEPOELE, OSS (NL)

(73) Assignee: CITRYLL B.V., OSS (NL)

(21) Appl. No.: 18/019,985

(22) PCT Filed: Aug. 5, 2021
(86) PCT No.: PCT/EP2021/071912

§ 371 Date: Feb. 6, 2023

(30) Foreign Application Priority Data
Aug. 7, 2020 (GB) 2012326.1

(51) Int. Cl. G01N 33/50

(52) U.S. Cl. CPC G01N 33/5047; G01N 2800/52

(57) ABSTRACT

The invention relates to methods of identifying human subjects who will respond to administration of an agent that inhibits the formation of neutrophil extracellular traps (NETs). Methods of measuring efficacy of treatment with an agent that inhibits the formation of NETs and methods of treatment comprising administration of such agents are also encompassed by the invention. Methods may comprise a cell fixation step.

### FIELD OF THE INVENTION

The invention relates to methods of identifying human subjects who will respond to administration of an agent that inhibits the formation of neutrophil extracellular traps (NETs). Methods of measuring efficacy of treatment with an agent that inhibits the formation of NETs, and methods of treatment comprising administration of such agents, are also encompassed by the invention.

### BACKGROUND TO THE INVENTION

Neutrophils are the most abundant type of leukocytes in human blood. They contribute to the first line of defence and use their extensive armoury to protect the host against infection. Neutrophils kill microbes via phagocytosis, generation of reactive oxygen species (ROS), or release of their granular contents. A more recently described antimicrobial function of neutrophils is neutrophil extracellular trap (NET) formation. NETs are structures composed of DNA, histones, and intracellular enzymes, which are released from granulocytes to immobilize and kill pathogens in blood and tissues. NETs confine and efficiently eliminate pathogens and have been shown to protect mice and humans against bacterial and fungal infections. Despite their importance in host defence, aberrant and prolonged NET release is associated with the pathophysiology of many acute and chronic inflammatory disorders.

In particular, incomplete clearance of NETs contributes to vascular injury, which leads to tissue damage and organ failure or even death. NETs have been shown to block tissue repair signals, leading to impaired wound healing in diabetes, while activation of the clotting system by NETs occludes blood vessels in thrombosis. In addition, antimicrobial proteins and histones that are present in NETs are highly cytotoxic and induce endothelial dysfunction in systemic lupus erythematosus (SLE), vasculitis and sepsis. Furthermore, NETs are a source of autoantigens and trigger autoimmunity, which is associated with the production of autoantibodies against various NET components in rheumatoid arthritis (RA), small-vessel vasculitis (SVV), antiphospholipid syndrome (APS) and SLE.

The formation of ROS via nicotinamide adenine dinucleotide phosphate (NADPH) oxidase complex, myeloperoxidase (MPO), or mitochondria, together with the translocation of neutrophil elastase (NE) and MPO to the nucleus, is a key mechanism of NET release. Moreover, conversion of arginine to citrulline on histones by peptidyl arginine deiminase 4 (PAD4) is necessary to promote chromatin decondensation and the subsequent release of NETs in the extracellular environment. Interestingly, pharmacological or genetic inhibition of PAD4 disrupts NET release and reduces pathology in various murine disease models, including atherosclerosis, inflammatory arthritis (IA) and SLE. Therefore, NETs are potential therapeutic targets for different acute and chronic inflammatory disorders.

Excessive release of neutrophil extracellular traps (NETs) is associated with disease severity and contributes to tissue injury, followed by severe organ damage. Pharmacological or genetic inhibition of NET formation and/or release reduces pathology in multiple inflammatory disease models, indicating that NETs are potential therapeutic targets. Therapeutic anti-citrullinated protein antibodies (tACPAs) have been shown to bind citrulline residues in the N-termini of histones 2A and 4 (Chirivi et al., 2013). Citrullinated histones are generated during NET formation, and treatment with tACPAs has been shown to prevent disease symptoms in various mouse models with plausible NET-mediated pathology, including inflammatory arthritis (IA), pulmonary fibrosis, inflammatory bowel disease and sepsis (Chirivi et al., 2013, Chirivi et al., 2020). tACPAs diminish NET release, and so inhibit NET formation.

There is a need to maximise the efficiency of treatment of patients suffering from NET-mediated or associated pathologies. Thus, there is a need to identify patients that will respond to treatment with agents that inhibit the formation of NETs. There is also a need to monitor the effectiveness of such treatments.

### SUMMARY OF THE INVENTION

The present invention encompasses:

A method of identifying a human subject who will respond to administration of an agent that inhibits the formation of NETs, the method comprising:
(i) measuring the level of NETs and/or preNETs in a blood sample from said subject; and
(ii) identifying the subject as responsive to administration of the agent when the percentage of NETs to total number of neutrophils, or the percentage of preNETs to total number of neutrophils, in said sample is greater than 1%.

The present invention also encompasses:

A method of monitoring the effectiveness of a method of treatment in a human subject comprising:
(i) administration of an agent that inhibits the formation of NETs, or a binding fragment thereof, to the human subject;
(ii) measuring the level of NETs, and/or preNETs in a blood taken sample from said subject after administration of the agent.

The present invention also encompasses:

A method of selecting a human subject for treatment with an agent that inhibits the formation of NETs, comprising:
(i) measuring the level of NETs and/or preNETs in a blood sample from said patient; and
(ii) identifying the human subject as responsive to administration of the agent when the percentage of NETs to total number of neutrophils, or the percentage of preNETs to total number of neutrophils, in said sample is greater than 1%; and
(iii) selecting the human subject for treatment with the agent based on the identification of the patient according to (ii).

In one preferred embodiment, a method of the present invention does not involve a fixation step. In an alternative preferred embodiment, a method of the present invention comprises a cell fixation step. For example, in some situations there may be an interval between sample recovery from a subject and analysis meaning that fixation may be preferably employed. In one embodiment, whole blood is fixed. In another embodiment, white cells isolated from whole blood are fixed. Where fixation is employed, any steps requiring that the cells be alive are performed prior to fixation.

The invention will now be described in more detail, by way of example and not limitation, and by reference to the accompanying drawings. Many equivalent modifications and variations will be apparent to those skilled in the art when given this disclosure. Accordingly, the exemplary embodiments of the invention set forth are considered to be illustrative and not limiting. Various changes to the described embodiments may be made without departing from the scope of the disclosure. All documents cited herein, whether supra or infra, are expressly incorporated by reference in their entirety.

The present disclosure includes the combination of the aspects and preferred features described except where such a combination is clearly impermissible or is stated to be expressly avoided. As used in this specification and the appended claims, the singular forms “a”, “an”, and “the” include plural referents unless the content clearly dictates otherwise. Thus, for example, reference to “an antibody” includes two or more such antibodies.

Section headings are used herein for convenience only and are not to be construed as limiting in any way.

### BRIEF DESCRIPTION OF THE FIGURES

FIG. 1: CIT-013 inhibits NET release. (A) Phase contrast, SYTOX™ Green and merged images from the live imaging fluorescence microscopy assay. Neutrophils from healthy volunteers have been treated with Ca-ionophore (A23187) in order to induce NET formation. cIgG-treated neutrophils released NETs in the extracellular environment starting as soon as 60 min after A23187 stimulation (purple arrows), while CIT-013 treatment resulted in pre-NETs (orange and red arrows). Plasma membrane rupture occurred upon NET release (yellow arrow), while the plasma membrane of CIT-013-treated pre-NETs is intact (blue arrows). Scale bars: 15 μm. (B) Quantification of the neutrophil and DNA surface of NETs and pre-NETs at t=210 min. n=6 donors in 3 independent experiments. Results depicted as means±SEM. ****P<0.0001 using unpaired Students t test.

FIG. 2: CIT-013 induces pre-NET accumulation. Quantification of NETs (>270 μm²; A) and pre-NETs (<270 μm²; C) over time based on live imaging fluorescence microscopic images. Neutrophils have been treated with Ca-ionophore (A23187) in order to induce NET formation. The amount of NETs or pre-NETs per time point were divided by the number of neutrophils on t=0 min and expressed as the percentage of NETs or pre-NETs. Additional calculations to determine the NET-inhibitory effect of CIT-013 (B) or the pre-NET accumulating effect of CIT-013 (D) at t=210 min. (E) SYTOX™ Green images over time of a SYTOX™ Green Bright pre-NET (top) and a SYTOX™ Green Dimm pre-NET (bottom) obtained during live imaging fluorescence microscopy. Scale bars: 5 μm. (F) Quantification of SYTOX™ Green Bright and Dimm pre-NETs at t=300 min after stimulation with A23187 and CIT-013 treatment. n=6 donors in 3 independent experiments in A, B, C, D, and F. Results depicted as means±SEM. *P<0.05, **P<0.01, ***P<0.001, and ****P<0.0001 using unpaired Students t test in A, C, and F or Kruskal-Wallis multiple comparison test in B and D.

FIG. 3: Binding characteristics of tACPA to healthy neutrophils. (A) Histograms that show fluorescence signal of HiLyte™Fluor 488, representing tACPA binding. The upper histogram shows that cIgG (white) and tACPA (blue) do not bind to non-activated neutrophils. The lower histogram shows an increased binding of tACPA to pre-NETs, compared to cIgG. The gate was used to determine the percentage of binding. (B). tACPA does bind to pre-NETs but does not bind to healthy neutrophils, monocytes, T cells, B cells, and NK cells and DCs (cIgG (n=4); tACPA (n=6)).

FIG. 4: Ex vivo pre-NET induction in whole blood. Whole blood was treated without antibody (No Ab), isotype control antibody (cIgG) or CIT-013 and stimulated with A23187 in order to induce NET formation. After 3 hours, leukocytes were fixed and permeabilized to determine CIT-013 binding to A23187-induced CD66+ leukocytes, which indicate pre-NETs. (A) Representative images of the gating strategy of cIgG-treated whole blood (top plots) and CIT-013-treated whole blood (bottom plots). Leukocytes positive for CD66b+(granulocytes) were selected to determine cIgG or CIT-013 binding, indicated by FITC positivity (FITC+; red squares). (B) Pooled data of two healthy donors, performed in a single experiment, showed increased percentage of A23187-induced pre-NETs. Results depicted as means±SEM.

FIG. 5: Forward side scatter dot plot of non-fixed whole blood. Shows results for FACS analysis of unfixed white blood cells isolated from whole blood of a healthy volunteer and stained with anti-CD15 antibody. The position of the granulocyte and monocyte populations are indicated.

FIG. 6: Summary of sample processing in Example 4. Provides a flow scheme of the different cell fixation approaches employed in Example 4.

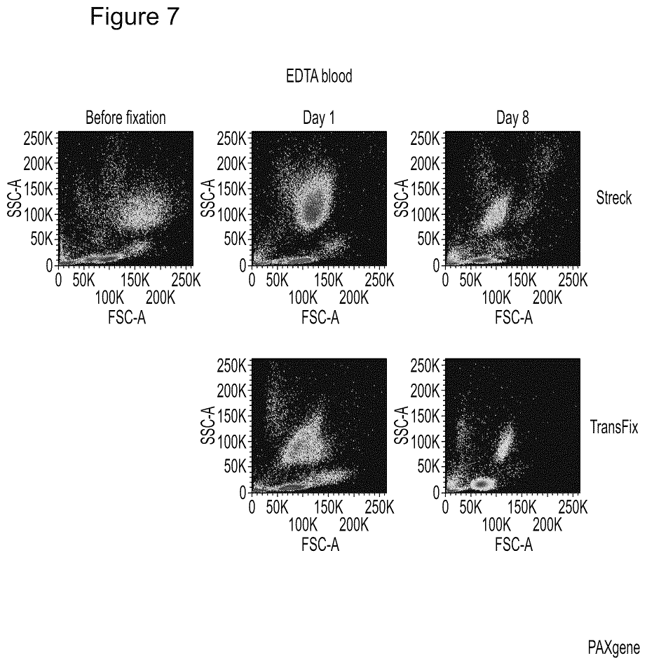

FIG. 7: Comparison of FACS results for samples prepared using different fixatives. Forward (x-axis, FSC-A) side scatter (y-axis, SSC-A) of fixed samples show changes in phenotypic characteristics in both EDTA blood and Lithium Heparin blood after 1 day and 8 days storage in Streck Cell Preservative, TransFix Cellular Antigen Stabilizing Reagent and PAXgene Blood DNA tube.

### DETAILED DESCRIPTION OF THE INVENTION

The present invention encompasses methods of detecting the response of a human subject to the administration of an agent that inhibits NET formation, or predicting the response of a human subject to administration of said agent, in order that to treat the human subject effectively. The purpose of the invention is to improve the treatment of NET-associated pathologies in a human subject in need thereof.

NET-associated pathologies can be defined as a disease or condition where the formation of NETs and NETosis is associated with the pathological state of the disease or condition. Whether or not NET formation and NETosis plays a role in the pathogenesis of the disease may be easily determined by a skilled person using routine tests available in the art. For example, these diseases may be characterized by the presence of NETs in relevant tissues.

Thus, in a preferred embodiment, the invention involves methods of treating a patient in need thereof with an agent that inhibits the formation of NETs, wherein the patient is suffering from a NET-associated pathology.

Examples of NET-associated pathologies include inflammatory conditions or diseases such as inflammatory diseases, autoimmune diseases, cancer, and organ-health after transplant.

“Inflammatory Conditions” or Inflammatory diseases” refers to any of a number of conditions or diseases, which are characterized by vascular changes: edema and infiltration of neutrophils (e.g., acute inflammatory reactions); infiltration of tissues by mononuclear cells; tissue destruction by inflammatory cells, connective tissue cells and their cellular products; and attempts at repair by connective tissue replacement (e.g., chronic inflammatory reactions). Such diseases include for instance inflammatory arthritis, including rheumatoid arthritis and osteoarthritis, SLE, idiopathic inflammatory myopathy, lupus, sepsis, vasculitis, small-vessel vasculitis (SVV), antiphospholipid syndrome (APS), multiple sclerosis, psoriatic arthritis, psoriasis, Alzheimer's disease, autoimmune hepatitis, juvenile idiopathic arthritis, ulcerative colitis, Sjögren's disease, Anti-phospholipid Syndrome, Bechet's disease, spondylitis, asthma, allergic rhinovirus exacerbated asthma, allergic asthma, spondyloarthropathy, multiple system atrophy, Parkinson's disease, Lewy body dementia, idiopathic pulmonary fibrosis, dry eye disease, uveitis, nongranulomatous uveitis, granulomatous uveitis, dermatitis, atopic dermatitis, inflammatory bowel disease and lung diseases such as COPD and bronchitis. Nongranulomatous uveitis can be associated with neutrophil dominant inflammation, granulomatous uveitis can be associated with macrophage dominant inflammation.

NETs play a role in autoimmune diseases pathology, including RA, SLE and vasculitis. The pathway by which the agents described herein improve the treatment of disease is likely via the inhibition of NET formation, the prevention of chromatin decondensation, the clearance of NET remnants, including toxic histones, and other autoantigens from tissue and circulation. For several autoimmune diseases it has been shown that the pathology improves in PAD knock-out models or in wild-type animals treated with a PAD inhibitor, meaning that there is a strong correlation with the amount of NETs and disease severity.

In a preferred embodiment, the diseases to be treated are NET-associated pathologies such as inflammatory conditions, autoimmune conditions, systemic lupus erythematosus (SLE), idiopathic inflammatory myopathy, lupus, sepsis, thrombosis, vasculitis, small-vessel vasculitis (SVV), antiphospholipid syndrome (APS), inflammatory arthritis, rheumatoid arthritis and osteoarthritis, psoriasis, Alzheimer's disease, autoimmune hepatitis, juvenile idiopathic arthritis, ulcerative colitis, Sjögren's disease, Anti-phospholipid Syndrome, Bechet's disease, spondylitis, spondyloarthropathy, multiple system atrophy, Parkinson's disease, Lewy body dementia, asthma, allergic rhinovirus exacerbated asthma, allergic asthma, cystic fibrosis, fibrosis and idiopathic pulmonary fibrosis, dry eye disease, uveitis, nongranulomatous uveitis, granulomatous uveitis, dermatitis, atopic dermatitis, inflammatory bowel disease, COPD, bronchitis, or other NET-associated pathologies such as wound healing in diabetes, cancer, cancer metastasis, transplant organ health in vivo or ex vivo and viral induced acute respiratory distress syndrome (ARDS).

### Agents of Interest to the Invention

The present inventors have discovered that agents that prevent chromatin decondensation in NET formation prevent NET release.

In particular, the inventors have shown herein that antibodies that specifically bind to a citrullinated epitope on deiminated human histone 2A and/or histone 4, termed therapeutic anti-citrullinated protein antibodies (tACPAs), prevent NET release. The NETosis pathway is halted at a stage before NETs are completely expelled from the neutrophils into the extracellular environment. Instead, NETs are retained inside the neutrophil or are partially expelled in a defined, contained area. The inventors have termed these neutrophils “preNETs”. The inventors have shown that the proportion of pre-NETs to NET in a sample is increased after administration of the tACPA, and that the proportion of NETs is decreased.

Chromatin decondensation is necessary for the subsequent release of NETs into the extracellular environment. Without being bound by theory, it is expected the action of the tACPA is associated with the prevention of chromatin decondensation in NET formation.

Thus, it is envisaged that administration of an agent that prevents chromatin decondensation in NET formation to a human subject with a NET-associated pathology would result in an initial increase in the proportion of preNETs to NETs in a sample taken from said subject, in comparison to the proportion seen in a sample taken before administration of the agent. It is expected that following the initial increase in the proportion of preNETs to NETs in the blood of said subject the administration would result in clearance of preNETs and NETs from the blood of said subject. Overall, the administration would result in a reduction in the level of NETs and preNETs in the blood of said subject, treating the NET-associated pathology in said subject. Thus, human subjects with NET-associated pathologies will be responsive to administration of an agent that inhibits NET formation by preventing chromatin decondensation.

The present invention encompasses methods of identifying a human subject who will respond to treatment with an agent which inhibits the formation of NETs. In a preferred embodiment, the present invention relates to methods of identifying a human subject who will respond to treatment with an agent which prevents chromatin decondensation, thus preventing NET formation and/or release.

The prevention of chromatin decondensation and/or prevention of NET release by the agents for use in the invention can be total or partial. For example, the agent for use in the invention may prevent chromatin decondensation and/or NET release by 10 to 50%, at least 50% or at least 70%, 80%, 90%, 95% or 99%. Prevention of chromatin decondensation and/or NET release can be measured by any suitable means, for example by measuring NETosis in vitro (van der Linden M et al., Sci. Rep. 2017).

The agent may be a small molecule, a chemotherapeutic agent, or an immunotherapeutic agent. Examples of agents include ROS inhibitors, JAK/STAT inhibitors, PAD4 inhibitors and Gasdermin D (GSDMD) inhibitors. Additional agents include Diphenyleneiodonium (DPI), Chlooramidin, Corticosteroids, C5a receptor antagonists, Necrostatin-1 (NEC-1), Necrosulfanomide (NSA), Vitamin D, Eculizumab, N-acetylcysteinine (NAC), MitoTEMPO, DNase I, Signal Inhibitory Receptor on Leukocytes-1 (SIRL-1), Tofacitinib, Metformin, Rituximab (RTX) in combination with Belimumab (BLM), Peptide Inhibitor of Complement C1 (PIC1), Hydroxychloroquine (HCQ), Anifrolumab, Calcineurin inhibitors, antimalarials, Baricitinib, mitochondrial ROS scavengers and drugs that disrupt neutrophil immunometabolism. Discussion of suitable agents can be found in Van Dam et al., 2018 Kidney Int. Rep. 4(2), pages 196-211 and Goel and Kaplan (2020) Curr Opin Rheumatol. 32(1), pages 64-70, 2020, which are herein incorporated by reference.

In a preferred embodiment the agent is one which inhibits the function of citrullinated histone 2A and/or histone 4. In a particularly preferred embodiment the agent is an antibody or binding fragment thereof that specifically binds to a citrullinated epitope on deiminated human histone 2A and/or histone 4.

Deimination of human histone 2A and 4 can be carried out by enzymes such as peptidylarginine deiminase (PAD), for example PAD2 and PAD4. The antibodies or binding fragments thereof for use in the invention may also specifically bind to a citrullinated epitope on human histone 3. The antibodies or binding fragments thereof for use in the invention may specifically bind to a citrullinated epitope on human histone 2A and/or histone 4 and/or histone 3.

The antibodies or binding fragments thereof for use in the present invention are disclosed herein by the primary amino acid sequence of their CDR regions. The antibodies or binding fragments thereof for use in the present invention are disclosed herein by the primary amino acid sequence of their heavy and light chains. In a particularly preferred embodiment the antibodies or binding fragments thereof for use in the present invention are as disclosed in WO2020/038963, incorporated herein by reference.

In a particularly preferred embodiment, the antibody for use in the invention is CIT-013, or a binding fragment thereof. The amino acid sequences of the VH and VL of the CIT-013 antibody or a binding fragment thereof are given in SEQ ID NOs: 11 and 16. The CDRs for the VH are shown in SEQ ID NOs: 1, 2 and 3. The CDRs for the VL are shown in SEQ ID NOs: 9, 4 and 5. The CIT-013 antibody may comprise a heavy chain constant region amino acid sequence comprising SEQ ID NO: 23 or 56, and the light chain constant region amino acid sequence of SEQ ID NO: 24. The CIT-013 antibody can also be referred to as hMQ22.101f/LC_41.

The CIT-013 antibody or binding fragment thereof described herein can be used to identify preNETs, or NETotic neutrophils, in a sample. The CIT-013 antibody or binding fragment thereof described herein can be used to identify preNETs, or NETotic neutrophils, in a sample, in order to diagnose a human subject with a NET-associated pathology as described herein. The CIT-013 antibody or binding fragment thereof of the invention can be used to determine the levels of preNETs, or NETotic neutrophils, in a sample. The CIT-013 antibody or binding fragment thereof of the invention can be used to determine the levels of preNETs, or NETotic neutrophils, in a sample, in order to diagnose a human subject with a NET-associated pathology as described herein, or to monitor the effectiveness of a treatment of said NET-associated pathology in said subject.

Identifying Human Subjects Responsive to Treatment with an Agent Described Herein and Predicting Responsiveness to Said Agent

Due to the analysis of the consequences of prevention of chromatin decondensation in NET formation conducted by the inventors, it is possible to characterise preNET and NET profiles in human subjects, in order to identify a subject responsive to treatment with agents that prevent of chromatin decondensation during NET formation. The agent can then be given to the subject in order to treat a NET-associated pathology in said subject.

It is also possible, given the teaching of the application, to monitor the effectiveness of said treatment by measuring the levels of preNETs, NETs or preNETs and NETs in a sample from the subject before and after administration with the agent.

It is also possible, given the teaching of the application to select a human subject for treatment with an agent that inhibits the formation of NETs, by measuring the level of NETs and/or preNETs in a blood sample from said subject, identifying the human subject as responsive to administration of the agent and selecting the human subject for treatment with the agent based on the identification of the subject.

PreNETs are defined herein as neutrophils that are in the process of NET formation, and can be considered to be NETotic neutrophils. In more detail, preNETs are defined herein as neutrophils with an amorphous decondensed nuclear structure containing citrullinated chromatin that still appears intracellularly, having a collapsed nuclear membrane and most likely a porous/punctured cell membrane. In the methods of the invention described herein preNETs are defined as having a cell area of less than approximately 270 μm² as measured by live imaging, preferably less than 260, 250 or 240 μm², as measured by live imaging.

NETs are defined herein as structures composed of DNA, histones, and intracellular enzymes, which immobilize and kill pathogens in blood and tissues. In the methods of the invention described herein NETs are defined as having a cell area of more than approximately 270 μm² as measured by live imaging, preferably more than 280, 290 or 300 μm² as measured by live imaging.

Methods to identify NETotic neutrophils by imaging, including by FACS, are known in the art. Examples of FACS methods for identifying NETotic neutrophils can be found in Gavillet M. et al., (2015), Manda-Handzlik A. et al., (2016), Lee K. H. et al., (2018), Zharkova O. et al., (2019) and Schneck E. et al., (2020), and which are all incorporated herein by reference. The inventors envisage the use of FACS to identify pre-NETs in samples, such as blood samples, from patients, individuals or human subjects. Pre-NETs can be distinguished from NETs according to the criteria set out herein. An example of a methodology is also set out in Example 2 of the present disclosure.

For example, according to Lee et al 2018, a sample from a healthy subject is expected to contain NETotic neutrophils at a concentration of less than 200 per μl of blood, as calculated by flow cytometry without use of PMA to stimulate NETosis. In contrast, a sample from a subject with a NET-associated pathology, such as sepsis or thrombosis, is expected to contain NETotic neutrophils at a concentration of at least 250 per μl, as calculated by flow cytometry without use of PMA to stimulate NETosis.

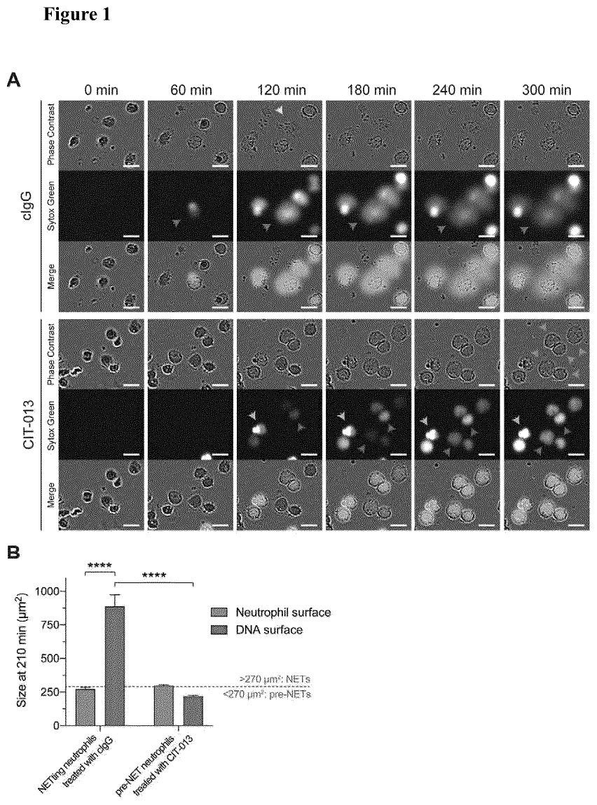

In the present invention a baseline level of pre-NETs in a sample from a healthy human subject can be considered to less than 200 per μl of blood, as calculated by flow cytometry, following the collection procedures of Lee et al. 2018 without use of PMA to stimulate NETosis. In contrast, sample from a human subject with a NET-associated pathology, who will thus respond to administration with an agent that prevents chromatin decondensation in NET formation, is expected to contain preNETs at a concentration of at least 250 per μl of blood, as calculated by flow cytometry, following the collection procedures of Lee et al. 2018 without use of PMA to stimulate NETosis. The authors of Lee et al. calculated that sepsis and thrombosis patients contain 3-5% NETotic neutrophils in the total CD15+ neutrophil population. From the data in Lee et al., it can be calculated that the percentage of NETotic neutrophils in a healthy human subject would be 0.084% of the total neutrophil population (percentage derived from data in FIG. 1C of Lee et al.).

Thus, in the present invention it is envisaged that a sample from a human subject who will respond to treatment with an agent that prevents chromatin decondensation will have a percentage of preNETs, or NETotic neutrophils, to total number of neutrophils of greater than 1%. In a preferred embodiment the percentage of preNETs, or NETotic neutrophils to total number of neutrophils is greater than 1.5%, 2%, 3%, 3.5%, 4%, 4.5%, 5%, 6%, 7%, 8%, 9%, 10% or more.

Thus, in the present invention it is envisaged that a sample from a human subject who will respond to treatment with an agent that prevents chromatin decondensation will have a percentage of NETs to total number of neutrophils of greater than 1%. In a preferred embodiment the percentage of NETs to total number of neutrophils is greater than 1.5%, 2%, 3%, 3.5%, 4%, 4.5%, 5%, 6%, 7%, 8%, 9%, 10% or more.

In a preferred embodiment, a human subject will be identified as being suitable for treatment with an agent which prevents chromatin decondensation during NET formation if they have a level of NETs, or NETs and/or preNETs in a blood sample of at least 1.5% of the total number of neutrophils identified. A subject is defined as having above a threshold minimum likelihood of having a clinical response to treatment with an agent for use in the invention where the level of NETs, or NETs and/or preNETs in a sample taken from said subject is greater than 1.5% of the total number of neutrophils.

The invention also encompasses methods of monitoring the effectiveness of a method of treatment in a human subject comprising administering an agent which inhibits NET formation and measuring the level of NETs, or NETs and/or pre-NETs in a blood sample from said subject. The invention also encompasses methods of monitoring the effectiveness of a method of treatment in a human subject comprising measuring the level of NETs, or NETs and/or pre-NETs in a blood sample from said subject, administering said agent and measuring the level of NETs, or NETs and/or pre-NETs in a blood sample taken from said subject after administration of the agent. It is envisaged that the administration of the agent will cause a decrease in the level of NETs and pre-NETs in said subject.

The invention also encompasses an agent for use in a method of monitoring the effectiveness of a method of treatment in a human subject comprising administering an agent which inhibits NET formation and measuring the level of NETs, or NETs and/or pre-NETs in a blood sample from said subject. The invention also encompasses an agent for use in methods of monitoring the effectiveness of a method of treatment in a human subject comprising measuring the level of NETs, or NETs and/or pre-NETs in a blood sample from said subject, administering said agent and measuring the level of NETs, or NETs and/or pre-NETs in a blood sample taken from said subject after administration of the agent. It is envisaged that the administration of the agent will cause a decrease in the level of NETs and pre-NETs in said subject.

Effectiveness of treatment is defined as the observation that administration of the agent has caused an improvement in the NET profile seen in blood samples from the subject. In more detail, it is expected that administration of an agent which inhibits NET formation by preventing chromatin decondensation will result in an initial increase in the proportion of preNETs to NETs in the blood of said subject, followed by clearance of both NETs and preNETs from the blood of said subject. The levels of preNETs and NETs can be calculated according to the criteria and methods described herein. The levels can also be validated by methods known in the art, such as live imaging.

The invention also encompasses a method of selecting a human subject for treatment with an agent that inhibits the formation of NETs, by measuring the level of NETs and/or preNETs in a blood sample from said subject, identifying the human subject as responsive to administration of the agent when the percentage of NETs to total number of neutrophils, or the percentage of preNETs to total number of neutrophils, in said sample is greater than 1%, and selecting the human subject for treatment with the agent based on the identification of the subject according to the above. In a preferred embodiment the percentage of preNETs, or NETotic neutrophils to total number of neutrophils is greater than 1.5%, 2%, 3%, 3.5%, 4%, 4.5%, 5%, 6%, 7%, 8%, 9%, 10% or more.

Thus, in the present invention it is envisaged that a sample from a human subject responsive to administration of the agent that prevents chromatin decondensation will have a percentage of NETs to total number of neutrophils of greater than 1%. In a preferred embodiment the percentage of NETs to total number of neutrophils is greater than 1.5%, 2%, 3%, 3.5%, 4%, 4.5%, 5%, 6%, 7%, 8%, 9%, 10% or more.

In the methods of the invention described herein a sample is considered to be a blood sample. In a preferred embodiment, the blood sample is a whole blood sample. In a preferred embodiment, the blood sample is a peripheral whole blood sample.

### Further Description of the Antibodies for Use in the Invention

The antibodies or binding fragments thereof for use according to the invention specifically bind to a citrullinated epitope on deiminated human histone 2A and/or histone 4. In a specific embodiment, the antibodies or binding fragments thereof according to the invention specifically bind to a citrullinated epitope on deiminated human histone 2A and/or histone 4, wherein the epitope comprises a peptide selected from the group consisting of SEQ ID NOs: 18, 19, 20, 21 and 22. The antibodies or binding fragments thereof may also bind to epitopes comprising the peptides of SEQ ID NO: 53 or 54.

The term “antibodies”, “antibody” or “binding fragment thereof” as used herein refers to a structure, preferably a protein or polypeptide structure, capable of specific binding to a target molecule often referred to as “antigen”.

The antibody molecule as employed herein refers to an antibody or binding fragment thereof. The term ‘antibody’ as used herein generally relates to intact (whole) antibodies i.e. comprising the elements of two heavy chains and two light chains. The antibody may comprise further additional binding domains for example as per the molecule DVD-Ig as disclosed in WO 2007/024715, or the so-called (FabFv)₂Fc described in WO2011/030107. Thus ‘antibody’ as employed herein includes mono-, bi-, tri- or tetra-valent full-length antibodies.

Binding fragments of antibodies include single chain antibodies (i.e. a full-length heavy chain and light chain); Fab, modified Fab, Fab′, modified Fab′, F(ab′)2, Fv, Fab-Fv, Fab-dsFv, single domain antibodies (e.g. VH or VL or VHH), scFv, mono-, bi-, tri- or tetra-valent antibodies, Bis-scFv, diabodies, tribodies, triabodies, tetrabodies and epitope-binding fragments of any of the above (see for example Holliger P and Hudson P J, 2005, Nat. Biotechnol., 23, 1126-1136; Adair J R and Lawson A D G, 2005, Drug Design Reviews—Online, 2, 209-217). The methods for creating and manufacturing these antibody fragments are well known in the art (see for example Verma R et al., 1998, J. Immunol. Methods, 216, 165-181). The Fab-Fv format was first disclosed in WO2009/040562 and the disulphide-stabilised versions thereof, the Fab-dsFv was first disclosed in WO2010/035012. Other antibody fragments for use in the present invention include Fab and Fab′ fragments. Multi-valent antibodies may comprise multiple specificities e.g. bispecific or may be monospecific.

An antibody or binding fragment thereof may be selected from the group consisting of single chain antibodies, single chain variable fragments (scFvs), variable fragments (Fvs), fragment antigen-binding regions (Fabs), recombinant antibodies, monoclonal antibodies, fusion proteins comprising the antigen-binding domain of a native antibody or an aptamer, single-domain antibodies (sdAbs), also known as VHH antibodies, nanobodies (Camelid-derived single-domain antibodies), shark IgNAR-derived single-domain antibody fragments called VNAR, diabodies, triabodies, Anticalins, aptamers (DNA or RNA) and active components or fragments thereof.

IgG1 (e.g. IgG1/kappa) antibodies having an IgG1 heavy chain and a light chain may advantageously be used in the invention. However, other human antibody isotypes are also encompassed by the invention, including IgG2, IgG3, IgG4, IgM, IgA1, IgA2, IgAsec, IgD and IgE in combination with a kappa or lambda light chain. Also, all animal-derived antibodies of various isotypes can be used in the invention. The antibodies can be full-size antibodies or antigen-binding fragments of antibodies, including Fab, F(ab′)2, single-chain Fv fragments, or single-domain VHH, VH or VL single domains.

The term: “specifically binds to citrulline” or “specifically binds to a citrullinated epitope” in this context means that the antibody or binding fragment thereof binds to a structure such as a peptide containing a citrulline residue whereas the antibody or binding fragment thereof binds less strongly or preferably not at all with the same structure containing an arginine residue instead of the citrulline residue. The term peptide should be interpreted as a structure that is capable of presenting the citrulline residue in the correct context for immunoreactivity with the antibodies or binding fragments thereof as described herein, preferably in the same context as it appears in the human or animal body, preferably in the context of a native polypeptide.

The antibodies or binding fragments thereof of the invention specifically bind to a citrullinated epitope on deiminated human histone 2A and/or histone 4. The binding of antibodies or binding fragments thereof to a citrullinated epitope on deiminated human histone 2A and/or histone 4 blocks NET formation. Citrullination of histones is associated with the formation of NETs.

Blocking of NET formation can be total or partial. For example, the antibody or binding fragment thereof of the invention may reduce NET formation from 10 to 50%, at least 50% or at least 70%, 80%, 90%, 95% or 99%. NET blocking can be measured by any suitable means, for example by measuring NETosis in vitro (van Linden et al., Sci. Rep. 2017).

The terms “binding activity” and “binding affinity” are intended to refer to the tendency of an antibody molecule to bind or not to bind to a target. Binding affinity may be quantified by determining the dissociation constant (Kd) for an antibody and its target. Similarly, the specificity of binding of an antibody to its target may be defined in terms of the comparative dissociation constants (Kd) of the antibody for its target as compared to the dissociation constant with respect to the antibody and another, non-target molecule.

Typically, the Kd for the antibody with respect to the target will be 2-fold, preferably 5-fold, more preferably 10-fold less than the Kd with respect to the other, non-target molecule such as unrelated material or accompanying material in the environment. More preferably, the Kd will be 50-fold less, even more preferably 100-fold less, and yet more preferably 200-fold less.

The value of this dissociation constant can be determined directly by well-known methods, and can be computed even for complex mixtures by methods such as those, for example, set forth in Caceci M S and Cacheris W P (1984, Byte, 9, 340-362). For example, the Kd may be established using a double-filter nitrocellulose filter binding assay such as that disclosed by Wong I and Lohman T M (1993, Proc. Natl. Acad. Sci. USA, 90, 5428-5432) or for example, by using Octet surface plasmon resonance.

One method for the evaluation of binding affinity for deiminated human histone 2A and/or histone 4 is by ELISA. Other standard assays to evaluate the binding ability of ligands such as antibodies towards targets are known in the art, including for example, Western blots, RIAs, and flow cytometry analysis. The binding kinetics (e.g. binding affinity) of the antibody also can be assessed by standard assays known in the art, such as surface plasmon resonance, for example by Biacore™ system analysis. Preferably the antibody or binding fragment thereof of the invention is a monoclonal antibody. Monoclonal antibodies are immunoglobulin molecules that are identical to each other and have a single binding specificity and affinity for a particular epitope. Monoclonal antibodies (mAbs) of the present invention can be produced by a variety of techniques, including conventional monoclonal antibody methodology, for example those disclosed in “Monoclonal Antibodies: a manual of techniques” (Zola H, 1987, CRC Press) and in “Monoclonal Hybridoma Antibodies: techniques and applications” (Hurrell J G R, 1982 CRC Press).

The antibody or binding fragment thereof for use according to the invention comprises a binding domain. A binding domain will generally comprise 6 CDRs (3 in case of VHH), three from a heavy chain and three from a light chain. In one embodiment the CDRs are in a framework and together form a variable region or domain. Thus in one embodiment an antibody or binding fragment comprises a binding domain specific for the antigen comprising a light chain variable region or domain and a heavy chain variable region or domain.

The residues in antibody variable domains are conventionally numbered according to IMGT (http://www.imgt.org). This system is set forth in Lefranc M P (1997, J, Immunol. Today, 18, 509). This numbering system is used in the present specification except where otherwise indicated.

The IMGT residue designations do not always correspond directly with the linear numbering of the amino acid residues. The actual linear amino acid sequence may contain fewer or additional amino acids than in the strict IMGT numbering corresponding to a shortening of, or insertion into, a structural component, whether framework or CDR, of the basic variable domain structure. The correct IMGT numbering of residues may be determined for a given antibody by alignment of residues of homology in the sequence of the antibody with a “standard” IMGT numbered sequence.

The CDRs of the heavy chain variable domain are located at residues 27-38 (CDR1 of VH), residues 56-65 (CDR2 of VH) and residues 105-117 (CDR3 of VH) according to the IMGT numbering system.

The CDRs of the light chain variable domain are located at residues 27-38 (CDR1 of VL), residues 56-65 (CDR2 of VL) and residues 105-117 (CDR3 of VL) according to the IMGT numbering system.

In a preferred embodiment, the CDR1 of the VL chain of the antibody or binding fragment thereof for use in the invention comprises or consists of the amino acid sequence QSL-X₁-D-X₂-D-X₃-KTY, wherein X₁ is V or L, X₂ is T, S, A or N and X₃ is G or A, provided that the amino acid sequence is not QSLLDSDGKTY (SEQ ID NO: 36) or QSLVDSDGKTY (SEQ ID NO: 37).

The amino acid sequences of the CDRs for the VH of a particular antibody or binding fragment thereof for use in the invention are shown in SEQ ID NOs: 1, 2 and 3. The CDRs 2 and 3 for the VL are shown in SEQ ID NOs: 4 and 5.

The amino acid sequences of the VH and VL of a particular antibody or binding fragment thereof for use in the invention are given in SEQ ID NOs: 11 and 13. The CDRs for the VH are shown in SEQ ID NOs: 1, 2 and 3. The CDRs for the VL are shown in SEQ ID NOs: 6, 4 and 5.

The amino acid sequences of the VH and VL of another antibody or binding fragment thereof for use in the invention are given in SEQ ID NOs: 11 and 14. The CDRs for the VH are shown in SEQ ID NOs: 1, 2 and 3. The CDRs for the VL are shown in SEQ ID NOs: 7, 4 and 5.

The amino acid sequences of the VH and VL of another antibody or binding fragment thereof for use in the invention are given in SEQ ID NOs: 11 and 15. The CDRs for the VH are shown in SEQ ID NOs: 1, 2 and 3. The CDRs for the VL are shown in SEQ ID NOs: 8, 4 and 5.

The amino acid sequences of the VH and VL of another antibody or binding fragment thereof for use in the invention are given in SEQ ID NOs: 11 and 16. The CDRs for the VH are shown in SEQ ID NOs: 1, 2 and 3. The CDRs for the VL are shown in SEQ ID NOs: 9, 4 and 5.

The amino acid sequences of the VH and VL of another antibody or binding fragment thereof for use in the invention are given in SEQ ID NOs: 11 and 17. The CDRs for the VH are shown in SEQ ID NOs: 1, 2 and 3. The CDRs for the VL are shown in SEQ ID NOs: 10, 4 and 5.

The amino acid sequences of the VH and VL of another antibody or binding fragment thereof for use in the invention are given in SEQ ID NOs: 12 and 13. The CDRs for the VH are shown in SEQ ID NOs: 1, 2 and 3. The CDRs for the VL are shown in SEQ ID NOs: 6, 4 and 5.

The amino acid sequences of the VH and VL of another antibody or binding fragment thereof for use in the invention are given in SEQ ID NOs: 12 and 14. The CDRs for the VH are shown in SEQ ID NOs: 1, 2 and 3. The CDRs for the VL are shown in SEQ ID NOs: 7, 4 and 5.

The amino acid sequences of the VH and VL of another antibody or binding fragment thereof for use in the invention are given in SEQ ID NOs: 12 and 15. The CDRs for the VH are shown in SEQ ID NOs: 1, 2 and 3. The CDRs for the VL chain are shown in SEQ ID NOs: 8, 4 and 5.

The amino acid sequences of the VH and VL of another antibody or binding fragment thereof for use in the invention are given in SEQ ID NOs: 12 and 16. The CDRs for the VH are shown in SEQ ID NOs: 1, 2 and 3. The CDRs for the VL are shown in SEQ ID NOs: 9, 4 and 5.

The amino acid sequences of the VH and VL of another antibody or binding fragment thereof for use in the invention are given in SEQ ID NOs: 12 and 17. The CDRs for the VH are shown in SEQ ID NOs: 1, 2 and 3. The CDRs for the VL are shown in SEQ ID NOs: 10, 4 and 5.

In an embodiment of the present invention, the antibody for use in the invention comprises the heavy chain variable domain amino acid sequence of SEQ ID NO: 11, the light chain variable domain amino acid sequence of SEQ ID NO: 16, a heavy chain constant region amino acid sequence comprising SEQ ID NO: 23 or 56, and the light chain constant region amino acid sequence of SEQ ID NO: 24.

In an embodiment of the present invention, the antibody for use in the invention comprises the heavy chain variable domain amino acid sequence of SEQ ID NO: 11, the light chain variable domain amino acid sequence of SEQ ID NO: 16, the heavy chain constant region amino acid sequence of SEQ ID NO: 23 or 56, and the light chain constant region amino acid sequence of SEQ ID NO: 24.

An antibody or binding fragment thereof for use in the invention may comprise one or more of the CDR sequences of any one of the specific antibodies as described above, except that the CDR1 of the VL is always present as either comprising or consisting of the amino acid sequence QSL-X₁-D-X₂-D-X₃-KTY, wherein X₁ is V or L, X₂ is T, S, A or N and X₃ is G or A, provided that the amino acid sequence is not QSLLDSDGKTY (SEQ ID NO: 36) or QSLVDSDGKTY (SEQ ID NO: 37), or either comprises or consists of SEQ ID NOs: 6, 7, 8, 9 or 10.

An antibody or binding fragment thereof for use in the invention may comprise one or more VH CDR sequences and alternatively or additionally one or more VL CDR sequences of said specific antibody, in addition to VL CDR1. An antibody or binding fragment thereof of the invention may comprise one, two or all three of the VH CDR sequences of a specific antibody or binding fragment thereof as described above and alternatively or additionally one, two or all three of the VL chain CDR sequences of said specific antibody or binding fragment thereof, including VL CDR1. An antibody or binding fragment thereof of the invention may comprises all six CDR sequences of a specific antibody or binding fragment as described above. By way of example, an antibody for use in the invention may comprise one of SEQ ID NO: 6, 7, 8, 9 or 10 and one or more of SEQ ID NOs: 1, 2, 3, 4 and 5.

In an embodiment of the invention, the CDR1 of the VL chain of the antibody or binding fragment thereof for use in the invention comprises or consists of the amino acid sequence QSL-Z₁-Z₂-Z₃-Z₄-Z₅-KTY, wherein Z₁ is V or L, Z₂ is D or E, Z₃ is T, S, A or N, Z₄ is D, E, S or A and Z₅ is G or A, provided that the amino acid sequence is not QSLLDSDGKTY (SEQ ID NO: 36) or QSLVDSDGKTY (SEQ ID NO: 37). The modified CDR1 of the VL chain of the antibody or binding fragment thereof for use in the invention may comprise or consist of SEQ ID NO: 6, 7, 8, 9, 10, 41, 42, 43, 44, 45, 46, 47, 48, 49, 50, 51 or 52. In an embodiment of the invention, the antibody for use in the invention may comprise one of SEQ ID NO: 6, 7, 8, 9, 10, 41, 42, 43, 44, 45, 46, 47, 48, 49, 50, 51 or 52, and one or more of SEQ ID NOs: 1, 2, 3, 4 and 5. In an embodiment of the invention, the antibody for use in the invention comprises one of SEQ ID NO: 6, 7, 8, 9, 10, 41, 42, 43, 44, 45, 46, 47, 48, 49, 50, 51 or 52, and all of SEQ ID NOs: 1, 2, 3, 4 and 5.

An antibody or binding fragment thereof for use in the invention may alternatively comprise a variant of one of these heavy chain variable domains or CDR sequences in CDR2 or 3 of the VL. For example, a variant may be a substitution, deletion or addition variant of any of the above amino acid sequences.

A variant antibody may comprise 1, 2, 3, 4, 5, up to 10, up to 20, up to 30 or more amino acid substitutions and/or deletions from the specific sequences and fragments discussed above, whilst maintaining the activity of the antibodies described herein. “Deletion” variants may comprise the deletion of, for example, 1, 2, 3, 4 or 5 individual amino acids or of one or more small groups of amino acids such as 2, 3, 4 or 5 amino acids. “Small groups of amino acids” can be defined as being sequential, or in close proximity but not sequential, to each other. “Substitution” variants preferably involve the replacement of one or more amino acids with the same number of amino acids and making conservative amino acid substitutions. For example, an amino acid may be substituted with an alternative amino acid having similar properties, for example, another basic amino acid, another acidic amino acid, another neutral amino acid, another charged amino acid, another hydrophilic amino acid, another hydrophobic amino acid, another polar amino acid, another aromatic amino acid, another aliphatic amino acid, another tiny amino acid, another small amino acid or another large amino acid. Some properties of the 20 main amino acids, which can be used to select suitable substituents, are as follows in Table 1 below:

TABLE 1

| | | | |
|---|---|---|---|
| Ala | aliphatic, hydrophobic, | Met | hydrophobic, neutral |
| | neutral | | |
| Cys | polar, hydrophobic, | Asn | polar, hydrophilic, |
| | neutral | | neutral |
| Asp | polar, hydrophilic, | Pro | hydrophobic, neutral |
| | charged (−) | | |
| Glu | polar, hydrophilic, | Gln | polar, hydrophilic, |
| | charged (−) | | neutral |
| Phe | aromatic, hydrophobic, | Arg | polar, hydrophilic, |
| | neutral | | charged (+) |
| Gly | aliphatic, neutral | Ser | polar, hydrophilic, neutral |
| His | aromatic, polar, hydrophilic, | Thr | polar, hydrophilic, neutral |
| | charged (+) | | |
| Ile | aliphatic, hydrophobic, | Val | aliphatic, hydrophobic, |
| | neutral | | neutral |
| Lys | polar, hydrophilic, | Trp | aromatic, hydrophobic, |
| | charged (+) | | neutral |
| Leu | aliphatic, hydrophobic, | Tyr | aromatic, polar, |
| | neutral | | hydrophobic |

Preferred “derivatives” or “variants” include those in which instead of the naturally occurring amino acid the amino acid, which appears in the sequence, is a structural analog thereof. Amino acids used in the sequences may also be derivatized or modified, e.g. labelled, providing the function of the antibody is not significantly adversely affected.

Derivatives and variants as described above may be prepared during synthesis of the antibody or by post-production modification, or when the antibody is in recombinant form using the known techniques of site-directed mutagenesis, random mutagenesis, or enzymatic cleavage and/or ligation of nucleic acids.

Preferably variant antibodies according to the invention have an amino acid sequence which has more than 60%, or more than 70%, e.g. 75 or 80%, preferably more than 85%, e.g. more than 90%, 95%, 96%, 97%, 98% or 99% amino acid identity to the VL and/or VH, or a fragment thereof, of an antibody disclosed herein. This level of amino acid identity may be seen across the full-length of the relevant SEQ ID NO sequence or over a part of the sequence, such as across 20, 30, 50, 75, 100, 150, 200 or more amino acids, depending on the size of the full-length polypeptide.

Preferably the variant antibodies comprise one or more of the CDR sequences as described herein.

In connection with amino acid sequences, “sequence identity” refers to sequences, which have the stated value when assessed using ClustalW (Thompson J D et al., 1994, Nucleic Acid Res., 22, 4673-4680) with the following parameters:

Pairwise alignment parameters—Method: slow/accurate, Matrix: PAM, Gap open penalty: 10.00, Gap extension penalty: 0.10;

Multiple alignment parameters—Matrix: PAM, Gap open penalty: 10.00, % identity for delay: 30, Penalize end gaps: on, Gap separation distance: 0, Negative matrix: no, Gap extension penalty: 0.20, Residue-specific gap penalties: on, Hydrophilic gap penalties: on, Hydrophilic residues: G, P, S, N, D, Q, E, K, R. Sequence identity at a particular residue is intended to include identical residues, which have simply been derivatized.

The present invention thus provides antibodies having specific VH and VL amino acid sequences and variants and fragments thereof, which maintain the function or activity of these VHs and VLs.

Accordingly, the present invention encompasses antibodies or binding fragments thereof comprising variants of the VH that retain the ability of specifically binding a citrullinated epitope on human deiminated human histone 2A and/or histone 4. A variant of the heavy chain may have at least 70%, at least 80%, at least 85%, at least 90%, at least 95%, at least 96%, at least 97%, at least 98% or at least 99% amino acid sequence identity to the unmodified VH. The variant of the VH may comprise a fragment of at least 7 amino acids of hVH22.101f or hVH22.101HC9 (SEQ ID NO: 11 and 12, respectively), wherein the antibody or binding fragment thereof retains the ability of being specifically reactive with a citrullinated epitope on deiminated human histone 2A and/or histone 4; or a variant of hVH22.101f or hVH22.101HC9 (SEQ ID NO: 11 and 12, respectively) having at least 70% amino acid sequence identity to a sequence of hVH22.101f or hVH22.101HC9 (SEQ ID NO: 11 and 12, respectively), wherein the antibody or binding fragment thereof retains the ability of being specifically reactive with a citrullinated epitope on deiminated human histone 2A and/or histone 4.

### Therapeutic Uses of the Agents Described Herein

The agents for use in accordance with the present invention maybe used in therapy. In therapeutic applications, the agents or compositions comprising said agents are administered to a subject already suffering from a disorder or condition, in an amount sufficient to cure, alleviate or partially arrest the condition or one or more of its symptoms. Such therapeutic treatment may result in a decrease in severity of disease symptoms, or an increase in frequency or duration of symptom-free periods. An amount adequate to accomplish this is defined as “therapeutically effective amount”. Effective amounts for a given purpose will depend on the severity of the disease or injury as well as the weight and general state of the subject. As used herein, the term “subject” includes any human.

In particular embodiments, the agents is an antibody or binding fragment thereof linked (directly or indirectly) to another moiety. The other moiety may be a therapeutic agent such as a drug. The other moiety may be a detectable label. The other moiety may be a binding moiety, such as an antibody or a polypeptide binding domain specific for a therapeutic target. The antibody or binding fragment thereof for use in the invention may be a bispecific antibody.

The therapeutic agent or a detectable label may be directly attached, for example by chemical conjugation, to an antibody or binding fragment thereof of the invention. Methods of conjugating agents or labels to an antibody are known in the art. For example, carbodiimide conjugation (Bauminger S and Wilchek M, 1980, Methods Enzymol., 70, 151-159) may be used to conjugate a variety of agents, including doxorubicin, to antibodies or peptides. The water-soluble carbodiimide, 1-ethyl-3-(3-dimethylaminopropyl) carbodiimide (EDC) is particularly useful for conjugating a functional moiety to a binding moiety.

Other methods for conjugating a moiety to antibodies can also be used. For example, sodium periodate oxidation followed by reductive alkylation of appropriate reactants can be used, as can glutaraldehyde cross-linking. However, it is recognised that, regardless of which method of producing a conjugate of the invention is selected, a determination must be made that the antibody maintains its targeting ability and that the functional moiety maintains its relevant function.

The therapeutic agent linked to the antibody may comprise a polypeptide or a polynucleotide encoding a polypeptide which is of therapeutic benefit. Examples of such polypeptides include anti-proliferative or anti-inflammatory cytokines.

The antibody may be linked to a detectable label. By “detectable label” it is meant that the antibody is linked to a moiety which, when located at the target site following administration of the antibody into a patient, may be detected, typically non-invasively from outside the body and the site of the target located. Thus, the antibody may be useful in imaging and diagnosis.

Typically, the label is or comprises a radioactive atom which is useful in imaging. Suitable radioactive atoms include 99mTc and 123I for scintigraphic studies. Other labels include, for example, spin labels for magnetic resonance imaging (MRI) such as 123I again, 131I, 111In, 19F, 13C, 15N, 17O, gadolinium, manganese or iron. Clearly, the sufficient amount of the appropriate atomic isotopes must be linked to the antibody in order for the molecule to be readily detectable.

The radio- or other labels may be incorporated in known ways. For example, the antibody, or fragment thereof, may be biosynthesised or may be synthesised by chemical amino acid synthesis using suitable amino acid precursors involving, for example, fluorine-19 in place of hydrogen. Labels such as 99mTc, 123I, 186Rh, 188Rh and 111In can, for example, be attached via cysteine residues in polypeptides. Yttrium-90 can be attached via a lysine residue. Preferably, the detectable label comprises a radioactive atom, such as, for example technetium-99m or iodine-123. Alternatively, the detectable label may be selected from the group comprising: iodine-123; iodine-131; indium-111; fluorine-19; carbon-13; nitrogen-15; oxygen-17; gadolinium; manganese; iron.

In one embodiment, an antibody of the invention is able to bind selectively to a directly or indirectly cytotoxic moiety or to a detectable label. Thus, in this embodiment, the antibody is linked to a moiety which selectively binds to a further compound or component which is cytotoxic or readily detectable.

An agent for use in the present invention, or a composition comprising said agent, may be administered via one or more routes of administration using one or more of a variety of methods known in the art. As will be appreciated by the skilled artisan, the route and/or mode of administration will vary depending upon the desired results. Preferred routes of administration for agents of the invention include intravenous, subcutaneous, intraocular, intramuscular, intradermal, intraperitoneal, spinal or other parenteral routes of administration, for example by injection or infusion. The phrase “parenteral administration” as used herein means modes of administration other than enteral and topical administration, usually by injection. Administration may be rectal, oral, ocular, topical, epidermal or by the mucosal route. Administration may be local, including peritumoral, juxtatumoral, intratumoral, to the resection margin of tumors, intralesional, perilesional, by intra cavity infusion, intravesicle administration, or by inhalation. In a preferred embodiment, the agent is administered intravenously or subcutaneously.

A suitable dosage of an agent, or for example an antibody or binding fragment thereof for use in the invention may be determined by a skilled medical practitioner. Actual dosage levels of the active ingredients in pharmaceutical compositions comprising the agent for use in the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular agent employed, the route of administration, the time of administration, the rate of excretion of the agent, the duration of the treatment, other drugs, compounds and/or materials used in combination with the particular compositions employed, the age, sex, weight, condition, general health and prior medical history of the patient being treated, and like factors well known in the medical arts.

A suitable dose of an antibody or binding fragment thereof for use in the invention may be, for example, in the range of from about 0.1 μg/kg to about 100 mg/kg body weight of the patient to be treated. For example, a suitable dosage may be from about 1 μg/kg to about 50 mg/kg body weight per week, from about 100 μg/kg to about 25 mg/kg body weight per week or from about 10 μg/kg to about 12.5 mg/kg body weight per week.

A suitable dosage may be from about 1 μg/kg to about 50 mg/kg body weight per day, from about 100 μg/kg to about 25 mg/kg body weight per day or from about 10 μg/kg to about 12.5 mg/kg body weight per day.

Dosage regimens may be adjusted to provide the optimum desired response (e.g. a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Dosage regimens may be adjusted as a consequence of the methods of the invention.

Agents may be administered in a single dose or in multiple doses. The multiple doses may be administered via the same or different routes and to the same or different locations. Alternatively, agents can be administered as a sustained release formulation, in which case less frequent administration is required. Dosage and frequency may vary depending on the half-life of the agent or for example the antibody in the patient and the duration of treatment that is desired. The dosage and frequency of administration can also vary depending on whether the treatment is prophylactic or therapeutic. In prophylactic applications, a relatively low dosage may be administered at relatively infrequent intervals over a long period of time. In therapeutic applications, a relatively high dosage may be administered, for example until the patient shows partial or complete amelioration of symptoms of disease.

Combined administration of two or more agents may be achieved in a number of different ways. In one embodiment, the antibody or binding fragment thereof and the other agent may be administered together in a single composition. In another embodiment, the antibody and the other agent may be administered in separate compositions as part of a combined therapy. For example, the antibody or binding fragment thereof may be administered before, after or concurrently with the other agent.

### Sample Preservation

In one embodiment, a method of the present invention does not comprise a cell fixation step. In some situations though there may be an interval between sample recovery from a subject and analysis meaning that fixation may be preferably employed. Neutrophils are fragile cells meaning that the ability to preserve neutrophils, particularly by fixing the cells, may be advantageous. In one preferred embodiment, a method of the present invention may include a fixation step. In one embodiment, whole blood is fixed. In another embodiment, white cells isolated from whole blood are fixed. In one embodiment, the fixative used is selected from Streck Cell Preservative (for instance Streck, cat.nr. 213350), Transfix Cellular Antigen Stabilizing Reagent (for instance Cytomark cat. nr.TFB-01-1) and PAXgene fixative (for instance BD Biosciences cat.nr. 761165). In a preferred embodiment the fixative used is Streck Cell Preservative. In another preferred embodiment, the fixative used is PAXgene fixative. In one embodiment, the fixative includes formaldehyde. In one embodiment the fixative does not include formaldehyde.

In one embodiment of the invention, the blood is harvested with EDTA. In another embodiment, the blood is harvested with lithium heparin. For example, in one preferred embodiment the blood is harvested with EDTA and fixed with Streck Cell Preservative. In another preferred embodiment, the blood is harvested with lithium heparin and fixed with Streck Cell Preservative. In another preferred embodiment, the blood is harvested with lithium heparin and fixed with Pax gene fixative.

In one embodiment, the length of time from fixing the cells to analysis may be kept below a maximum. For instance, in one embodiment, the interval between fixation and analysis may be from 4 hours to 2 weeks. In one embodiment, the interval is from 12 hours to 10 days. In one interval, the sample is analysis within 10 days of fixation. In one embodiment, cells are analysed within 8 days of fixation. In another embodiment, analysis is performed within 4 days, 3 days, 2 days or 1 day from fixation. In a preferred embodiment, the sample is analysed within 36 hours of fixation. In another, the sample is analysed within about one day of harvesting. In another embodiment, a method of the present invention comprises fixing the samples and then storing the sample at room temperature. In another embodiment, the method comprises fixing the sample and then storing at from 2 to 8° C. once fixed.

In one embodiment, a method of the present invention comprises collecting blood with EDTA or Lithium Heparin, with fixation using Streck fixative and performing analysis within 12 to 36 hours. In a preferred embodiment, analysis is performed within about a day. In another embodiment, a method of the present invention comprises collecting blood with Lithium Heparin, and fixation using PAX gene fixative. In a preferred embodiment analysis is then performed within 12 to 36 hours. In a more preferred embodiment analysis is performed within 1 day of storage.

It may be that where a method of the invention comprises steps where the neutrophils are ideally alive, such steps are performed prior to fixation. For example, where a method of the present invention comprises treatment with an activator in one embodiment that is performed prior to fixation. In another embodiment, where the method of the invention involves the effect on NET formation, the fixation may be performed after the step allowing NET formation.

A method of the invention may comprise FACS analysis and in one embodiment staining with anti-CD15 antibody is performed with FACS analysis. For example, staining with anti-CD15 and FACS analysis may be used to check that the granulocyte population has not changed significantly due to fixation in terms of the results seen in FACS analysis, such as via forward scatter. A method of the invention may comprise analysis for CD15 positive cells to check the percentage of granulocytes.

### EXAMPLES

### Example 1—CIT-013 Inhibits NET Release Through Accumulation of Pre-NETs

Citrullinated histone H2A and H4 are identified epitopes of tACPA, including development candidate CIT-013, and have been shown to be specific targets for the inhibition of NET release. The aim of this study is to determine CIT-013's NET-inhibitory mechanism at the cellular level. With the use of live imaging fluorescence microscopy, we were able to visualize and quantify CIT-013's NET-inhibitory capacity over time. We observed that CIT-013 inhibits NET release by inducing pre-NET accumulation. This means that CIT-013 is able to block the release of NETs in the extracellular environment and thereby reduces the release of noxious triggers that are proinflammatory, toxic, and autoantigenic.

Citrullinated histone H2A and H4 are the identified targets of tACPA and are present in and on neutrophils in which the NETosis pathway has been activated (Neeli et al., 2008). Previous in vitro and in vivo experiments have demonstrated that: 1) tACPAs including CIT-013 bind to citrullinated histone H2A and H4 present in pre-NETs as well as expelled NETs; and 2) tACPAs inhibit NET release in vitro and in vivo (Chirivi et al., 2020). However, so far, CIT-013's exact mechanism of action (MoA) of inhibiting NET release remains to be unravelled. Therefore, the aim of this study is to decipher CIT-013's NET-inhibitory mechanism at the cellular level. With the use of live imaging fluorescence microscopy, we were able to visualize and quantify CIT-013's NET-inhibitory capacity over time.

### Methods

### Data and Statistical Analysis

Microscopic images were analyzed with ImageJ software (version 2.0.0-rc-69/1.52p). All statistical analyses were performed using GraphPad Prism software version 8.3.0. Results were reported as mean±standard error of the mean (SEM) and were considered significant at P<0.05. The normal distribution of each data set was assessed with the Shapiro-Wilk normality test. The comparison of two groups was performed by a two-tailed unpaired Students t test, while the comparison of three groups was performed by Kruskal-Wallis multiple comparison test. n is indicated in the figure legends.

### Neutrophil Isolation

Neutrophils were isolated from blood of healthy volunteers and treated with isotype control (cIgG) or CIT-013 before stimulation with calcium ionophore (A23187). NET release was visualized by means of live imaging fluorescence microscopy for 300 min. Microscopic images were analyzed with ImageJ software using a semi-automatic surface-based analyzing approach to quantify the percentage of pre-NETs and NETs (van der Linden et al., 2017).

Blood samples from healthy volunteers (HVs) were obtained through informed consent in accordance with the Declaration of Helsinki. Blood was collected in VACUETTE® Lithium Heparin tubes and neutrophils were isolated with Ficoll Paque Plus density gradient centrifugation followed by dextran/saline sedimentation with 6% dextran T500 in a 0.9% NaCl solution. Red blood cells were lysed with an ammonium-chloride-potassium lysis buffer (155 mM NH₄Cl, 10 mM KHCO₃ and 0.1 mM EDTA; pH=7.4) for 5 min at room temperature. Finally, neutrophils were resuspended in RPMI 1640 containing GlutaMAX and supplemented with 10% FBS and 1× penicillin-streptomycin.

### Live Imaging Immunofluorescence NET Assay

The live imaging immunofluorescence NET assay and quantification was performed as described before with minor differences. In short, neutrophils were resuspended in RPMI 1640 (without phenol red (-pr)) supplemented with 2% FBS, 1× Penicillin-Streptomycin, and 10 mM HEPES (referred to as RPMI-pr 2% hereafter). In experiments in which F(ab′)2 and Fab fragments were used, intracellular nuclear DNA of neutrophils was stained with Nuclear-ID Red DNA stain (1000× diluted; Enzo Life Sciences, ENZ-52406) for 15 min at 37° C., before seeding neutrophils in 0.001% poly-L-lysine (Sigma Aldrich, P4832) pre-coated clear bottom 96-well plates (Corning, Costar 3603). Neutrophils were treated with 25 μg/ml full-length CIT-013, 17.7 μg/ml CIT-013 F(ab′)2 fragments, or 8.5 μg/ml CIT-013 Fab fragments (similar molarity for full-length CIT-013, CIT-013 F(ab′)2, and CIT-013 Fab fragments) before stimulation with 5-25 μM A23187. A23187 was resuspended in RPMI-pr 2% containing 160 nM SYTOX™ Green (Life Technologies, S7020). NET release was recorded at 37° C. and 5% CO₂ in the IncuCyte ZOOM platform with a 20× objective during a period of 300 min. Every 30 min, a set of three images (Phase contrast and Sytox Green (Exc/Em: 504/523)) was taken.

### Semi-Automatic Quantification Approach

This NET quantification approach is a derivative of a previously described method (van der Linden et al., 2017). For quantification of NETs, images were processed with Fiji software (version 2.0.0-rc-69/1.52p). Image stacks were created and converted to 8-bit greyscale using a specific macro.

To count the number of neutrophils a macro was created that performed the following steps: The t=0 min phase contrast images were exposed to the “Find Edges” and “Smooth” analysis. Subsequently thresholding was done with “Default dark” logarithm using 122-255 settings, followed by “Fill Holes” analysis and “Watershed” segmentation to separate particles that touch. Particles with a size of >45 μm2 were counted as neutrophils.

To count pre-NETs and NETs, a macro was created that performed the following steps: Thresholding of SYTOX™ Green image stacks was performed with “RenyiEntropy dark” logarithm using 16-255 settings, followed by “Watershed” segmentation. Particles with a size of 10-270 μm² were counted as pre-NETs, whereas particles with a size of >270 μm² were counted as NETs. The number of pre-NETs or NETs were divided by the total number of neutrophils in t=0 min to calculate percentages.

### Results

### Visualization of CIT-013-Mediated NET Inhibition

We performed a live imaging fluorescence microscopy assay to monitor CIT-013-mediated NET inhibition over time. NETs were visualized with SYTOX™ Green, a cell impermeable dye that binds to DNA when it is exposed to the extracellular environment. Neutrophils treated with cIgG before stimulation with A23187 showed plasma membrane rupture (FIG. 1A; yellow arrow) and NET release (FIG. 1A; purple arrows). The NET surface (the surface of the secreted DNA that is stained with SYTOX™ Green staining) did increase over time as a result of diffusion. In contrast, neutrophils treated with CIT-013 showed intracellular SYTOX™ Green staining (FIG. 1A; orange and red arrows) and an intact plasma membrane (FIG. 1A; blue arrows) but did not expel NETs in the extracellular environment. These neutrophils are called pre-NETs and are positive for SYTOX™ Green because the plasma membrane of neutrophils becomes permeable due to NETosis pathway activation. Additional analysis measuring the surface of the neutrophil as well as its DNA confirmed that the DNA from pre-NETs is intracellular. Indeed, the surface of DNA in pre-NETs is smaller (<270 μm²) than the surface of the neutrophil itself, demonstrating that the DNA is intracellular (FIG. 1B). In contrast, the surface of the NET DNA is significantly larger (>270 μm²) than the surface of the neutrophil itself implying that the NETs are extracellular.

### CIT-013 Blocks NET Release by Inducing Pre-NET Accumulation

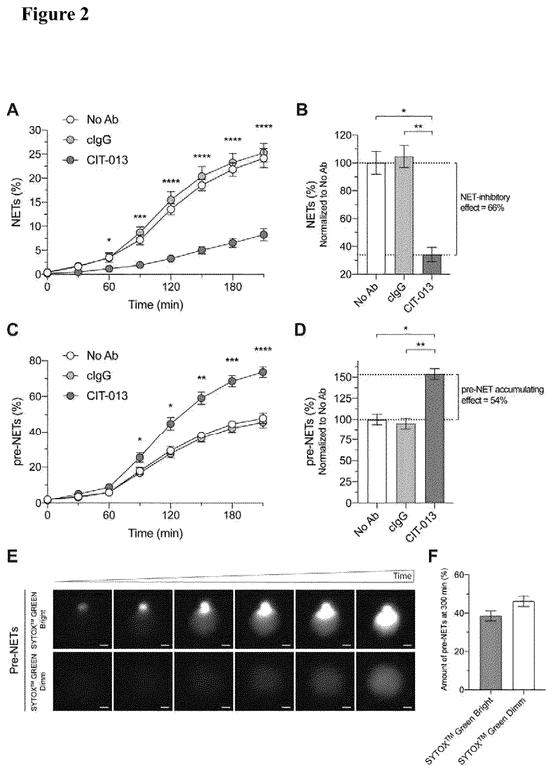

Using a semi-automatic surface-based analyzing approach we were able to distinguish between NETs (>270 μm²) and pre-NETs (<270 μm²). Quantification demonstrated that non-treated (No Ab) as well as cIgG-treated neutrophils released NETs as fast as 60 min upon stimulation with A23187 and that maximum NET release (25%) was reached after 210 min (FIG. 2A). As expected, CIT-013-treated neutrophils showed a significantly reduced amount of NET release over time, 8% NETs after 210 min which translates to NET-inhibitory effect of 66% compared to No Ab (FIG. 2B). CIT-013-mediated NET-inhibition resulted in pre-NET accumulation. Maximum accumulation was reached after 210 min (FIG. 2C) where the number of pre-NETs were increased with 54% compared to No Ab treatment (FIG. 2D).

Microscopy revealed two forms of CIT-013-induced pre-NETs based on the SYTOX™ Green intensity, these were named SYTOX™ Green Dimm (FIG. 1A; red arrows and FIG. 2E; lower images) and SYTOX™ Green Bright pre-NETs (FIG. 1A; orange arrows and FIG. 2E; upper images). The numbers of these two forms of pre-NETs at 300 min after A23187 stimulation and CIT-013 treatment is similar (FIG. 2F), however we noted phenotypical differences. Indeed, SYTOX™ Green Dimm pre-NETs contained decondensed DNA in a perfectly round shape, whereas SYTOX™ Green Bright pre-NETs contained decondensed DNA with a small protrusion. Interestingly, the SYTOX™ Green staining of this small protrusion was more intense and did spread all over the nucleus over time. These microscopic images suggest that the plasma membrane of SYTOX™ Green Bright pre-NETs ruptured somewhere in time, leading to the extrusion of some decondensed DNA to the extracellular space, which was immediately blocked by CIT-013. In SYTOX™ Green Dimm pre-NETs we hypothesize that CIT-013 enters the cells through active or passive transport and blocks further DNA decondensation from the inside out.

CIT-013 is able to inhibit NET release by inducing pre-NET accumulation. NET release appears to be polarized and happening on one side of the neutrophil.

### Example 2—the Binding Characteristics of tACPA to Healthy Leukocytes

CIT-013 is a first in class humanized monoclonal antibody, a so called therapeutic anti-citrullinated protein antibody (tACPA), targeting neutrophil extracellular traps (NETs) with a high potential for treating multiple inflammatory mediated diseases with high medical need (e.g. systemic lupus erythematosus (SLE), vasculitis, idiopathic pulmonary fibrosis (IPF), ulcerative colitis (DC)). Neutrophils are a part of the innate immune system, and inhibition of NET formation by CIT-013 inhibits the release of neutrophil decondensed DNA coated with pro-inflammatory proteins, citrullinated autoantigens as well as the release of toxic histones from the neutrophil. In this study a precursor molecule of CIT-013 was used, named MQ22.101j/e, which differs in 1 amino acid in its light chain CDR1 compared to CIT-013.

Citrullinated histone H2A and H4 are the identified targets of tACPA and are present in and on neutrophils in which the NETosis pathway has been activated. Previous in vitro and in vivo experiments have demonstrated that tACPA binds to citrullinated histone H2A and H4 present in pre-NETs as well as expelled NETs. However, so far, it is unknown whether healthy neutrophils and other leukocytes also contain binding targets of tACPA. To further determine the safety profile of tACPAs, their binding to healthy leukocytes was determined in order to exclude the existence of epitopes or receptors and hence potential side-effects. Therefore, the aim of this study is to exclude that tACPA binds to healthy leukocytes. With the use of CD markers, we were able to specifically determine tACPA binding characteristics to separate types of leukocytes, i.e. T cells, B cells, monocytes, natural killer (NK) cells, dendritic cells (DCs), and neutrophils.

### Methods

### Data and Statistical Analysis

Flow cytometry data was analyzed with FlowLogic software (version 7.2.1). All statistical analyses were performed using GraphPad Prism software version 8.3.0. Results were reported as mean±standard error of the mean (SEM) and were considered significant at P<0.05. The normal distribution of each data set was assessed with the D'Agostino-Pearson omnibus normality test, but the number of sample units, n, was too small to test for normal distribution. The comparison of two groups was performed by unpaired nonparametric two-tailed Kolmogorov-Smirnov test.

### Experimental Methods

Blood samples from healthy volunteers (HVs) were obtained from employees of Absano in Oss, The Netherlands or from the Sanquin blood bank in Nijmegen, The Netherlands. All HVs gave informed consent in accordance with the Declaration of Helsinki. Blood was collected in VACUETTE® Lithium Heparin tubes and peripheral blood mononuclear cells (PBMCs) were isolated with Ficoll Paque Plus density gradient centrifugation. Neutrophils were isolated with Ficoll Paque Plus density gradient centrifugation followed by dextran/saline sedimentation with 6% dextran T500 in a 0.9% NaCl solution. Red blood cells were lysed with an ammonium-chloride-potassium lysis buffer (155 mM NH₄Cl, 10 mM KHCO₃ and 0.1 mM EDTA; pH=7.4) for 5 min at room temperature. Finally, neutrophils and PBMCs were resuspended in RPMI 1640 containing GlutaMAX and supplemented with 10% FBS and 1× penicillin-streptomycin.

To study the binding characteristics of tACPA to healthy PBMCs and neutrophils, 2×10⁵ cells per well were used to block Fc receptors with Human Trustain FcX for 20 min at room temperature. Subsequently, staining was performed for 45 min at room temperature with 0.17 μg/ml anti-human CD3-PerCP/Cy5.5, 1 μg/ml anti-human CDllc-Brilliant Violet 510, 0.33 (μg/ml anti-human CD14-APC, 0.17 μg/ml anti-human CD20-Pacific Blue, 0.083 μg/ml anti-human CD45-APC/Cy7, 0.083 μg/ml anti-human CD66b-PE, and 6.25 μg/ml hMQ22.101j/e-HiLyte™Fluor 488 (tACPA). MQR2.201a-Hilyte™Fluor 488 was used as isotype control (clgG). As a positive control, neutrophils were stimulated with 25 μM calcium ionophore A23187 for 60 min and 37° C. after which neutrophils were fixed with fixative solution and stained as described before. Measurements were performed with the BD FACS Canto II using FACS Diva software. The protocol can be thus be carried out as below:
1. Obtain blood from healthy volunteers (HVs) or patients.

### Positive Control:

2. Transfer 1 ml blood to a fresh FACS tube.
3. Stimulate blood from HVs with 4 μM calcium ionophore for 4 hours.
4. Transfer 100 μl blood from HVs, patients, or calcium ionophore stimulated blood to a fresh FACS tube.
5. Add 2 μl Fc receptor block.
6. Incubate for 10 min at room temperature.
7. Add primary antibody (i.e. CIT-013).
8. Incubate for 30 min at room temperature.
9. Add secondary antibody (i.e. goat-anti-human-Alexa Fluor 488).
10. Incubate for 30 min at room temperature.
11. Add 1 ml red blood cell lysis buffer.
12. Incubate for 10 min at room temperature.
13. Add 1 ml PBS.
14. Centrifuge for 10 min at 200 g and room temperature.
15. Discard supernatant and wash with 1 ml PBS.
16. Centrifuge for 10 min at 200 g and room temperature.
17. Discard supernatant and 100 μl Zombie NIR cell viability stain.
18. Incubate for 15 min at room temperature.
19. Centrifuge for 10 min at 200 g and room temperature.
20. Discard supernatant and wash with 1 ml PBS containing 5% BSA and 0.1% NaN₃.
21. Centrifuge for 10 min at 200 g and room temperature.
22. Discard supernatant and resuspend in 250 μl PBS containing 5% BSA and 0.1% NaN₃.
23. Measure with BD FACS Canto II.

### Results and Conclusion

CD markers were used to determine T cells (CD3+), B cells (CD20+), monocytes (CD14+), neutrophils (CD66b+), NK cells and DCs (CDllc+) in the isolated fractions of whole blood. Neutrophils that were stimulated with 25 μM A23187 for 60 min to activate their NETosis pathway (pre-NETs) resulting in the presence of citrullinated histone H2A and H4. These pre-NETs were used as positive control for tACPA binding. Binding of tACPA to the above described leukocytes, as well as pre-NETs, were determined by detecting the HiLyte™Fluor 488 fluorescence signal.

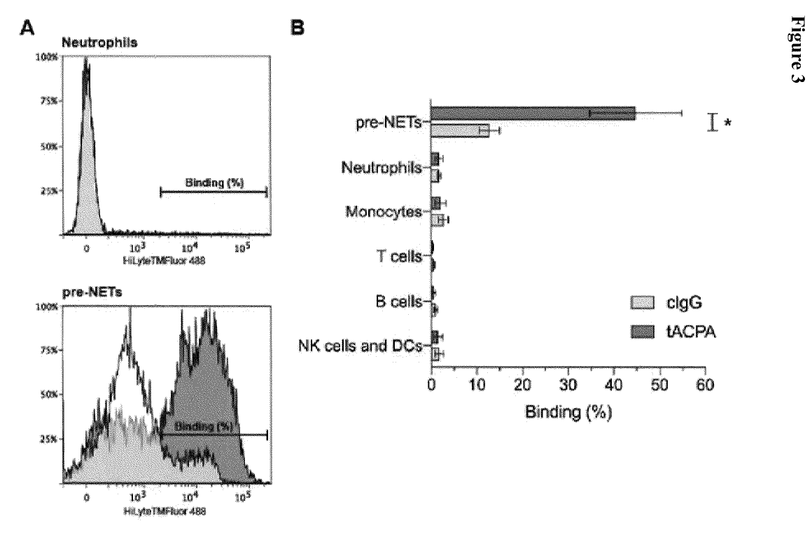

As expected, no binding of isotype control (clgG; white) and tACPA (blue) was observed to non-activated neutrophil (FIG. 3A; upper histogram), while tACPA, compared to clgG, showed increased binding to pre-NETs (FIG. 3A; lower histogram). We observed that tACPA does not bind to any type of healthy leukocyte (FIG. 3B), meaning that healthy leukocytes do not contain unknown binding targets of tACPA on their cellular membrane.

### Example 3—Ex Vivo Pre-NET Induction in Whole Blood

### Method

Blood samples from healthy volunteers (HVs) were obtained from the Sanquin blood bank in Nijmegen, The Netherlands and collected in VACUETTE® Lithium Heparin tubes. All HVs gave informed consent in accordance with the Declaration of Helsinki. One milliliter blood was transferred to FACS tubes before adding 25 μg/ml CIT-013 or isotype control antibody (cIgG). No antibody was used as negative control. To induce pre-NET formation, 12.5 μM A23187 was added to the blood and incubated for 3 hours at 37° C. After incubation, blood was centrifuged for 15 min at 190×g at room temperature (RT). Plasma was transferred to a fresh 1.5 ml tube, centrifuged for 10 min at 1500×g at RT to remove platelets and stored at −80° C. Plasma can be used for an ELISA approach to detect NETs.

The mix of red blood cells and leukocytes was diluted with 750 μl PBS followed by dextran/saline sedimentation with 2 ml 6% dextran T500 in a 0.9% NaCl solution. Left over red blood cells were lysed with 10 ml ammonium-chloride-potassium lysis buffer (155 mM NH₄Cl, 10 mM KHCO₃ and 0.1 mM EDTA; pH=7.4) for 5 min at RT. After two washing steps with PBS containing 1% v/w BSA and 0.01% v/w NaN₃ (FACS buffer), leukocytes were treated with 500 μl 1× fixation/permeabilization solution for 40 min at RT in the dark. Fixed and permeabilized leukocytes were treated with Human Trustain FcX (50× diluted in permeabilization buffer) for 20 min at RT and incubated with a mix of antibodies containing goat-anti-human IgG-Alexa Fluor 488 (400× diluted in permeabilization buffer) and mouse-anti-human CD66b (400× diluted in permeabilization buffer) for 45 min at RT. Subsequently, leukocytes were washed twice with permeabilization buffer and once with FACS buffer before acquisition was performed with the BD FACS Canto II using FACS Diva software.

### Results

As we showed before, neutrophils release NETs upon stimulation with A23187 in vitro which is inhibited with CIT-013. The NET-inhibitory capacity of CIT-013 occurs because CIT-013 binds neutrophils with an activated NETosis pathway (characterized by their citrullinated histones and further appointed as pre-NETs). Thus, pre-NETs could be indicated by CIT-013 binding.

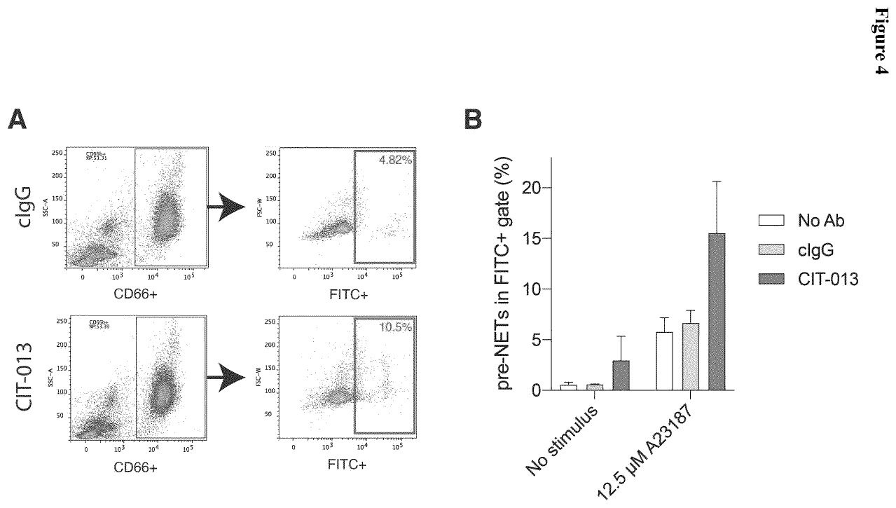

We here demonstrate a flow cytometry-based assay that determines pre-NETs in whole blood. CIT-013 was added to whole blood from healthy volunteers before adding A23187 to induce NET release. After leukocyte isolation, we used CD66b to select for granulocytes followed by goat-anti-human IgG-Alexa Fluor 488 to detect CIT-013 binding (FITC+ gate red squares; FIG. 4A). Pooled data from two healthy volunteers demonstrates the presence of ˜15% pre-NETs when whole blood was stimulated with A23187 in the presence of CIT-013 (FIG. 4B). The absence of any antibody (No Ab) and cIgG were used as negative control and shows ˜5% pre-NETs.

### Conclusion

We have developed a flow cytometry-based assay that is able to detect pre-NETs in whole blood ex vivo.

### Example 4: Comparison of Fixation Conditions

### Introduction

Given that it may not be always possible for logistical reasons to analyse samples on the day of harvesting blood, the ability to preserve samples in the same state as at the time of blood withdrawal is important, particularly as neutrophils are fragile cells that lyse easily. For that reason, the ability of different fixative solutions to preserve samples was assessed.

Three fixative cellular stabilization solutions were therefore tested to determine if neutrophils can survive in a fixation solution and maintain their immunophenotypical properties. The three preservatives assessed were:
1) Streck Cell Preservative (Streck, cat.nr. 213350): a general easy to use reagent that contains the anticoagulant K₂EDTA and a cell preservative in a liquid medium. (Streck makes no claims regarding the presence or absence of formaldehyde). Product information indicates that samples can be stored up to 7 days at 2-10° C. for BD Biosciences Systems.
2) TransFix Cellular Antigen Stabilizing Reagent (Cytomark cat. nr.TFB-01-1): a green liquid containing paraformaldehyde and other cell preservatives. Product information indicates that samples can be stored for 14 days at 2-8° C. and up to 4 days at 18-25° C.
3) PAXgene Blood DNA tube (BD Biosciences cat.nr. 761165): a proprietary blend of reagents without formaldehyde that both prevents blood coagulation and stabilizes white blood cells. Product information indicates samples can be stored up to 10-14 days at room temperature.

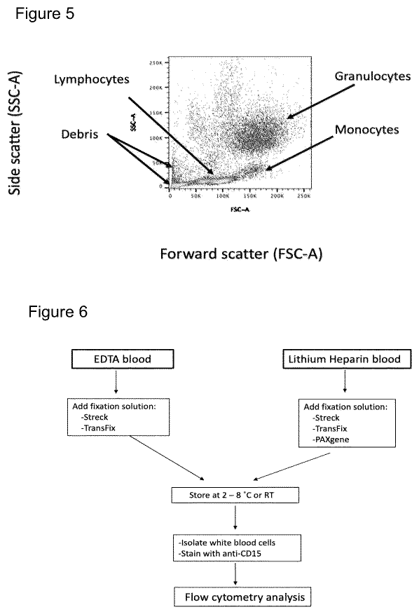

Neutrophils are the most abundant cells in the granulocyte fraction besides eosinophils, basophils and mast cells. The relative number of granulocytes (CD15 positive cells) were determined with flow cytometry analysis before and after fixation. In addition, phenotypic changes, including size and granularity, were analysed using forward side scatter with the results obtained shown in FIG. 5.

### Blood Processing and Neutrophil Fixation

Blood samples from healthy volunteers (HVs) were obtained via venepuncture from Sanquin (Dutch Bloedbank) at Nijmegen, The Netherlands. The healthy volunteers gave informed consent in accordance to the Declaration of Helsinki. Blood was collected in K₃EDTA tubes or Lithium Heparin tubes. Blood was mixed with Streck Cell Preservative (1 mL blood was added to 1 mL Streck Cell Preservative), TransFix Cellular Antigen Stabilizing Reagent (200 μL TransFix solution was added to 1 mL blood) and PAXgene Blood ccfDNA tube (2.5 mL blood was added to 1 PAXgene tube). Samples were mixed by inverting the tubes at least 10 times and stored. Streck and TransFix samples were stored at 2-8° C., PAXgene samples were stored at room temperature.

Aliquots of whole blood (100 μL) and fixed blood (volumes were corrected for dilution with fixation solution: 120 μL for TransFix, 200 μL for Streck and 100 μL for PAXgene samples) were prepared in FACS tubes after 1 day and 8 days storage at 2-8° C. or room temperature. Blood cell lysis was performed twice on all samples by addition of 2 mL ACK lysis buffer (155 mM NH₄Cl, 10 mM KHCO₃ and 0.1 mM EDTA, pH 7.4) and incubated for a few minutes at room temperature, followed by centrifugation for 5 minutes at 350×g. The supernatant was discarded. The pelleted white blood cell fractions were washed with 2 mL FACS buffer (1% BSA+0.1% NaN₃ in PBS) and centrifuged for 5 minutes at 350×g at room temperature. The supernatant was discarded, and the cell pellet was resuspended in 300 μL FACS buffer.

### Sample Preparation and Staining

The resuspended white blood cells fractions were stained with a granulocyte marker (mouse anti-human CD15-PerCP, Biolegend cat. nr. 323018) at a concentration of 6.6 μg/mL. The solutions were gently mixed and incubated for at least 30 minutes at room temperature. Sample analysis was performed on the FACS Canto II (Becton Dickinson) using FACS Diva software.

### Results and Conclusions

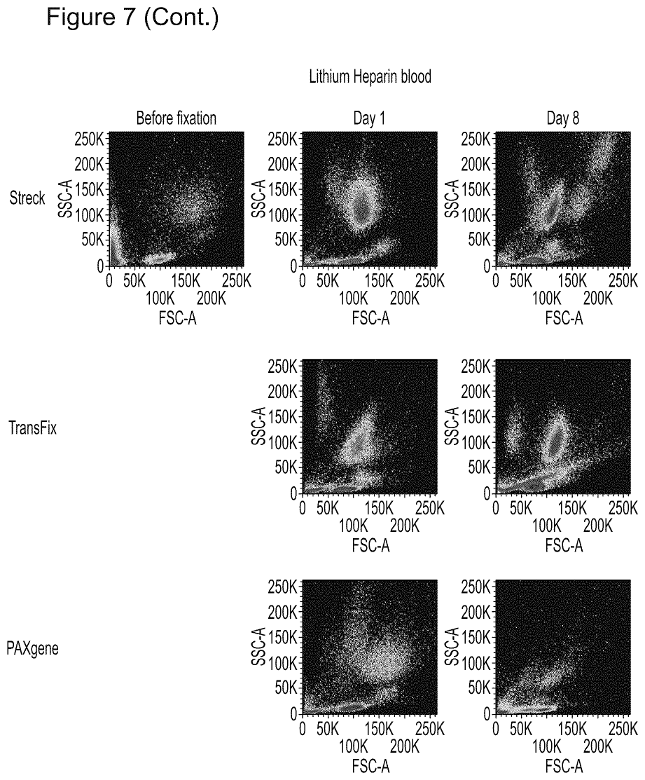

Granulocytes (CD15 positive cells) were detected in all samples, even after 8 days of fixation but the relative number of CD15 positive cells differed for each fixation tube (Table 2 below). A large decrease in CD15 positive cells after 8 days was determined in the PAXgene tubes. The relative number of CD15 positive cells in some fixed samples was higher than before fixation, which could be due to the elevated level of dying CD15 negative cells. The phenotypic characteristics of the granulocytes after fixation has changed based on the forward side scatter (FIG. 7), with granulocytes in Streck and PAXgene tubes after 1 day that most closely match with healthy neutrophils.

TABLE 2
Relative percentage of CD15 positive cells (granulocytes) in the fixed samples
after 1 day and 8 days storage at 2-8° C. (in Streck Cell Preservative and
TransFix Cellular Antigen Stabilizing Reagent) or RT (in PAXgene Blood DNA tube).

| | Before | Day 1 | | | Day 8 | | |
|---|---|---|---|---|---|---|---|
| | fixation | Streck | TransFix | PAXgene | Streck | TransFix | PAXgene |
| EDTA blood | 44.5% | 60.4% | 41.2% | ND | 33.8% | 18.3% | ND |
| Lithium | 37.1% | 57.4% | 41.7% | 42.0% | 38.6% | 40.8% | 12.6% |
| Heparin blood | | | | | | | |

(ND = Not Determined)

In conclusion it was demonstrated that granulocytes could still be detected by flow cytometry after fixation and 1 and 8 days of storage. The best conditions, based on percentage CD15+ cells (Table 2) as well as phenotypic characteristics (FIG. 7), were EDTA or Lithium Heparin blood with Streck fixative after 1 day of storage or Lithium Heparine blood with PAX gene fixative after 1 day of storage. Eight days of fixation seemed to harm the neutrophils and caused change in the forward side scatter.

### BRIEF DESCRIPTION OF THE SEQUENCE LISTING

### Antibody Nomenclature

CDR=complementarity-determining region.
VH=heavy chain variable domain.
VL=light chain variable domain.
CH=heavy chain constant domain.
CL=light chain constant domain.
msVH22.101=mouse VH of therapeutic antibody.
msVL22.101=mouse VL of therapeutic antibody.
hVH22.101x=humanized VH of therapeutic antibody, ‘x’ refers to the heavy chain.
hVL22.101y=humanized VL of therapeutic antibody, ‘y’ refers to the light chain.
hVH22.101(HC)x=humanized VH of therapeutic antibody, ‘(HC)x’ refers to the heavy chain.
hVL22.101(LC)y=humanized VL of therapeutic antibody, ‘(LC)y’ refers to the light chain.
hMQ22.101x/y=humanized therapeutic antibody, ‘x’ refers to the heavy chain, ‘y’ refers to the light chain.
hMQ22.101(HC)x/(LC)y=humanized therapeutic antibody, ‘(HC)x’ refers to the heavy chain, ‘(LC)y’ refers to the light chain.
hMQ22.101f/LC_41=the preferred antibody CIT-013

| SEQ ID | |
|---|---|
| NO | Name |
| 1 | CDR1 of msVH22.101 and hVH22.101(HC)x |
| 2 | CDR2 of msVH22.101 and hVH22.101(HC)x |
| 3 | CDR3 of msVH22.101 and hVH22.101(HC)x |
| 4 | CDR2 of msVL22.101 and hVL22.101(LC)y |
| 5 | CDR3 of msVL22.101 and hVL22.101(LC)y |
| 6 | CDR1 of hVL22.101LC17 |
| 7 | CDR1 of hVL22.101LC21 |
| 8 | CDR1 of hVL22.101LC27 |
| 9 | CDR1 of hVL22.101LC41 |
| 10 | CDR1 of hVL22.101LC42 |
| 11 | hVH22.101f |
| 12 | hVH22.101HC9 |
| 13 | hVL22.101LC17 |
| 14 | hVL22.101LC21 |
| 15 | hVL22.101LC27 |
| 16 | hVL22.101LC41 |
| 17 | hVL22.101LC42 |
| 18 | SEQ ID NO 1 from WO2016092082 |
| 19 | SEQ ID NO 2 from WO2016092082 |
| 20 | Shortened SEQ ID NO 2 from WO2016092082 |
| 21 | Peptide no 4 (human histone 2A) |
| | (SEQ ID NO 24 from WO2011070172) |
| 22 | Peptide no 6 (human histone 2A) |
| | (SEQ ID NO 26 from WO2011070172) |
| 23 | Human heavy chain constant domain of IgG1 |
| 24 | Human kappa chain constant domain |
| 25 | msVH22.101 |
| 26 | hVH22.101j |
| 27 | hVH22.101HC7 |
| 28 | hVH22.101HC8 |
| 29 | hVH22.101HC10 |
| 30 | msVL22.101 |
| 31 | hVL22.101e |
| 32 | hVL22.101g |
| 33 | hVL22.101h |
| 34 | hVL22.101i |
| 35 | hVL22.101j |
| 36 | CDR1 of msVL22.101 and hVL22.101g |
| 37 | CDR1 of hVL22.101e |
| 38 | CDR1 of hVL22.101h |
| 39 | CDR1 of hVL22.101i |
| 40 | CDR1 of hVL22.101j |
| 41 | CDR1 of hVL22.101LC16 |
| 42 | CDR1 of hVL22.101LC19 |
| 43 | CDR1 of hVL22.101LC20 |
| 44 | CDR1 of hVL22.101LC22 |
| 45 | CDR1 of hVL22.101LC23 |
| 46 | CDR1 of hVL22.101LC24 |
| 47 | CDR1 of hVL22.101LC25 |
| 48 | CDR1 of hVL22.101LC26 |
| 49 | CDR1 of hVL22.101LC37 |
| 50 | CDR1 of hVL22.101LC38 |
| 51 | CDR1 of hVL22.101LC39 |
| 52 | CDR1 of hVL22.101LC40 |
| 53 | msFibβ XG (SEQ ID NO 37 from WO2011070172) |
| 54 | msVim XS/XL (SEQ ID NO 38 from WO2011070172) |
| 55 | Region around CDR2 of msVL22.101 and |
| | hVL22.101(LC)y |
| 56 | Heavy chain constant domain of hCH22.101f |

Sequence listing

SEQ ID NO: 1-CDR1 of msVH22.101 and hVH22.101(HC)x
GYTFTNYG
SEQ ID NO: 2-CDR2 of msVH22.101 and hVH22.101(HC)x
INTYSGEA
SEQ ID NO: 3-CDR3 of msVH22.101 and hVH22.101(HC)x
LRGYTYQSFDEGGDY
SEQ ID NO: 4-CDR2 of msVL22.101 and hVL22.101(LC)y
LVS
SEQ ID NO: 5-CDR3 of msVL22.101 and hVL22.101(LC)y
WQGTHEPYT
SEQ ID NO: 6-CDR1 of hVL22.101LC17
QSLLDTDGKTY
SEQ ID NO: 7-CDR1 of hVL22.101LC21
QSLLDSDAKTY
SEQ ID NO: 8-CDR1 of hVL22.101LC27
QSLLDTDAKTY
SEQ ID NO: 9-CDR1 of hVL22.101LC41
QSLLDADGKTY
SEQ ID NO: 10-CDR1 of hVL22.101LC42
QSLLDNDGKTY
SEQ ID NO: 11-hVH22.101f
RIQLVQSGAEVKKPGASVKVSCKASGYTFTNYGMHWVRQAPGQGLEWMGWINTYSGEATYAQKFQGRVTMTRDTSISTAYM
ELSRLRSDDTAVYYCLRGYTYQSFDEGGDYWGQGTLVTVSS
SEQ ID NO: 12-hVH22.101HC9
RIQLVQSGAEVKKPGASVKVSCKASGYTFTNYGMHWVRQAPGQGLEWMGWINTYSGEATYVDDFQGRVTMTRDTSISTAYM
ELSRLRSDDTAVYYCLRGYTYQSFDEGGDYWGQGTLVTVSS
SEQ ID NO: 13-hVL22.101LC17
DVVMTQSPLSLPVTLGQPASISCRSSQSLLDTDGKTYLNWFQQRPGQSPRRLIYLVSKLDSGVPDRFSGSGSGTDFTLKIS
RVEAEDVGVYYCWQGTHEPYTFGQGTKLEIK
SEQ ID NO: 14-hVL22.101LC21
DVVMTQSPLSLPVTLGQPASISCRSSQSLLDSDAKTYLNWFQQRPGQSPRRLIYLVSKLDSGVPDRFSGSGSGTDFTLKIS
RVEAEDVGVYYCWQGTHEPYTFGQGTKLEIK
SEQ ID NO: 15-hVL22.101LC27
DVVMTQSPLSLPVTLGQPASISCRSSQSLLDTDAKTYLNWFQQRPGQSPRRLIYLVSKLDSGVPDRFSGSGSGTDFTLKIS
RVEAEDVGVYYCWQGTHEPYTFGQGTKLEIK
SEQ ID NO: 16-hVL22.101LC41
DVVMTQSPLSLPVTLGQPASISCRSSQSLLDADGKTYLNWFQQRPGQSPRRLIYLVSKLDSGVPDRFSGSGSGTDFTLKIS
RVEAEDVGVYYCWQGTHEPYTEGQGTKLEIK
SEQ ID NO: 17-hVL22.101LC42
DVVMTQSPLSLPVTLGQPASISCRSSQSLLDNDGKTYLNWFQQRPGQSPRRLIYLVSKLDSGVPDRFSGSGSGTDFTLKIS
RVEAEDVGVYYCWQGTHEPYTFGQGTKLEIK
SEQ ID NO: 18-SEQ ID NO 1 from WO2016092082 (used in Example 1/7) from histone 2A
SGXGKQGGKARA
Where X is citrulline
SEQ ID NO: 19-SEQ ID NO 2 from WO2016092082, (used in Example 7) from histone 4
SGXGKGGKGLGKGGAKRHRKVLR
Where X is citrulline
SEQ ID NO: 20-Shortened SEQ ID NO 2 from WO2016092082 (used in Example 7) from histone 4
SGXGKGGKGLGK
Where X is citrulline
SEQ ID NO: 21-Peptide no 4 (human histone 2A) (SEQ ID NO 24 from WO2011070172)
QFPVGXVHRLLR
Where X is citrulline
SEQ ID NO: 22-Peptide no 6 (human histone 2A) (SEQ ID NO 26 from WO2011070172)
VHRLLXKGNYSE
Where X is citrulline
SEQ ID NO: 23-Human heavy chain constant domain of IgG1
ASTKGPSVFPLAPSSKSTSGGTAALGCLVKDYFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTISKAKGQPREPQVYTLPPSRDELTK
NQVSLTCLVKGFYPSDIAVEWESNGQPENNYKTTPPVLDSDGSFFLYSKLTVDKSRWQQGNVFSCSVMHEALHNHYTQKSL
SLSPG
SEQ ID NO: 24-Human kappa chain constant domain
RTVAAPSVFIFPPSDEQLKSGTASVVCLLNNFYPREAKVQWKVDNALQSGNSQESVTEQDSKDSTYSLSSTLILSKADYEK
HKVYACEVTHQGLSSPVTKSFNRGEC
SEQ ID NO: 25-ms VH22.101
RIQLVQSGPELKKPGEAVKISCKASGYTFTNYGMHWMKQTPGKDFRWMGWINTYSGEATYVDDFKGRFAFSLGTSASTAYL
QINNLKNDDTATYFCLRGYTYQSFDEGGDYWGQGTALTVSS
SEQ ID NO: 26-hVH22.101j
QVQLVQSGAEVKKPGASVKVSCKASGYTFTNYGMHWVRQAPGQGLEWMGWINTYSGEATYAQKFQGRVTMTRDTSISTAYM
ELSRLRSDDTAVYYCLRGYTYQSFDEGGDYWGQGTLVTVSS
SEQ ID NO: 27-hVH22.101HC7
QVQLVQSGAEVKKPGSSVKVSCKASGYTFTNYGMHWVRQAPGQGLEWMGWINTYSGEATYAQKFQGRVTITADESTSTAYM
ELSSLRSEDTAVYYCLRGYTYQSFDEGGDYWGQGTLVTVSS
SEQ ID NO: 28-hVH22.101HC8
QVQLVQSGAEVKKPGSSVKVSCKASGYTFTNYGMHWVRQAPGQGLEWMGWINTYSGEATYVDDFQGRVTITADESTSTAYM
ELSSLRSEDTAVYYCLRGYTYQSEDEGGDYWGQGTLVTVSS
SEQ ID NO: 29-hVH22.101HC10
QVQLVQSGAEVKKPGASVKVSCKASGYTFTNYGMHWVRQAPGQGLEWMGWINTYSGEATYVDDFQGRVTMTRDTSISTAYM
ELSRLRSDDTAVYYCLRGYTYQSFDEGGDYWGQGTLVTVSS
SEQ ID NO: 30-msVL22.101
DVVMTQTPLILSVTTGQPASISCKSSQSLLDSDGKTYLNWLFQRPGQSPKRLIYLVSKLDSGVPDRFTGSGSGTDFTLKIS
RVEAEDLGIYYCWQGTHFPYTFGGGTNLEIK
SEQ ID NO: 31-hVL22.101e
DVVMTQSPLSLPVTLGQPASISCRSSQSLVDSDGKTYLNWFQQRPGQSPRRLIYLVSKLDSGVPDRFSGSGSGTDFTLKIS
RVEAEDVGVYYCWQGTHEPYTFGQGTKLEIK
SEQ ID NO: 32-hVL22.101g
DVVMTQSPLSLPVTLGQPASISCRSSQSLLDSDGKTYLNWFQQRPGQSPRRLIYLVSKLDSGVPDRFSGSGSGTDFTLKIS
RVEAEDVGVYYCWQGTHEPYTFGQGTKLEIK
SEQ ID NO: 33-hVL22.101h
DVVMTQSPLSLPVTLGQPASISCRSSQSLVASDGKTYLNWFQQRPGQSPRRLIYLVSKLDSGVPDRFSGSGSGTDFTLKIS
RVEAEDVGVYYCWQGTHEPYTFGQGTKLEIK
SEQ ID NO: 34-hVL22.101i
DVVMTQSPLSLPVTLGQPASISCRSSQSLVESDGKTYLNWFQQRPGQSPRRLIYLVSKLDSGVPDRFSGSGSGTDFTLKIS
RVEAEDVGVYYCWQGTHEPYTFGQGTKLEIK
SEQ ID NO: 35-hVL22.101j
DVVMTQSPLSLPVTLGQPASISCRSSQSLVSSDGKTYLNWFQQRPGQSPRRLIYLVSKLDSGVPDRFSGSGSGTDFTLKIS
RVEAEDVGVYYCWQGTHEPYTFGQGTKLEIK
SEQ ID NO: 36-CDR1 of msVL22.101 and hVL22.101g
QSLLDSDGKTY
SEQ ID NO: 37-CDR1 of hVL22.101e
QSLVDSDGKTY
SEQ ID NO: 38-CDR1 of hVL22.101h
QSLVASDGKTY
SEQ ID NO: 39-CDR1 of hVL22.101i
QSLVESDGKTY
SEQ ID NO: 40-CDR1 of hVL22.101j
QSLVSSDGKTY
SEQ ID NO: 41-CDR1 of hVL22.101LC16
QSLLESDGKTY
SEQ ID NO: 42-CDR1 of hVL22.101LC19
QSLLDSEGKTY
SEQ ID NO: 43-CDR1 of hVL22.101LC20
QSLLDSSGKTY
SEQ ID NO: 44-CDR1 of hVL22.101LC22
QSLLESEGKTY
SEQ ID NO: 45-CDR1 of hVL22.101LC23
QSLLESSGKTY
SEQ ID NO: 46-CDR1 of hVL22.101LC24
QSLLESDAKTY
SEQ ID NO: 47-CDR1 of hVL22.101LC25
QSLLDTEGKTY
SEQ ID NO: 48-CDR1 of hVL22.101LC26
QSLLDTSGKTY
SEQ ID NO: 49-CDR1 of hVL22.101LC37
QSLLDSAGKTY
SEQ ID NO: 50-CDR1 of hVL22.101LC38
QSLLESAGKTY
SEQ ID NO: 51-CDR1 of hVL22.101LC39
QSLLDAEGKTY
SEQ ID NO: 52-CDR1 of hVL22.101LC40
QSLLDNEGKTY
SEQ ID NO: 53-msFibß XG (SEQ ID NO 37 from WO2011070172)
EPTDSLDAXGHRPVDRR
Where X is citrulline
SEQ ID NO: 54-ms Vim XS/XL (SEQ ID NO 38 from WO2011070172)
YVTXSSAVXLXSSVP
Where X is citrulline
SEQ ID NO: 55-Region around CDR2 of msVL22.101 and hVL22.101(LC)y
LVSKLDS
SEQ ID NO: 56-Heavy chain constant domain of hCH22.101f
ASTKGPSVFPLAPSSKSTSGGTAALGCLVKDYFPEPVTVSWNSGALISGVHTFPAVLQSSGLYSLSSVVTVPSSSLGTQTY
ICNVNHKPSNTKVDKKVEPKSCDKTHTCPPCPAPELLGGPSVFLFPPKPKDTLMISRTPEVTCVVVDVSHEDPEVKFNWYV
DGVEVHNAKTKPREEQYNSTYRVVSVLIVLHQDWLNGKEYKCKVSNKALPAPIEKTISKAKGQPREPQVYTLPPSRDELTK
NQVSLTCLVKGFYPSDIAVEWESNGQPENNYKTTPPVLDSDGSFFLYSKLTVDKSRWQQGNVFSCSVMHEALHNHYTQKSL
SLSPGK

### REFERENCES

Adair J. R. and Lawson A. D. G., (2005). Drug Design Reviews—Online, 2, pages 209-217.
Bauminger S. and Wilchek M. (1980). Methods Enzymol., 70, pages 151-159.
Caceci M. S. and Cacheris W. P. (1984). Byte, 9, pages 340-362.
Chirivi, R. G. S., et al. (2013). Anti-citrullinated protein antibodies as novel therapeutic drugs in rheumatoid arthritis. J. Clin. Cell Immunol. 6, pages 1-13.
Chirivi R. G. S et al. (2020). Therapeutic ACPA inhibits NET formation: a potential therapy for neutrophil-mediated inflammatory diseases. Cellular & Molecular Immunology. https://doi.org/10.1038/s41423-020-0381-3
Gavillet M., Martinod K., Renella R. et al. (2015). Flow cytometric assay for direct quantification of neutrophil extracellular traps in blood samples. Am J Hematol., 90, pages 1155-1158.
Goel R. R. and Kaplan M. J. (2020) Deadliest catch: neutrophil extracellular traps in autoimmunity. Curr Opin Rheumatol. 32(1), pages 64-70.
Holliger P. and Hudson P. J., (2005). Nat. Biotechnol., 23, pages 1126-1136.
Hurrell J. G. R (1982). Monoclonal Hybridoma Antibodies: techniques and applications CRC Press
Kraaij T. et al. (2016). A novel method for high-throughput detection and quantification of neutrophil extracellular traps reveals ROS-independent NET release with immune complexes. Autoimmun. Rev. 15, 577-584.
Lee K. H. et al. (2018). Quantification of NETs-associated markers by flow cytometry and serum assays in patients with thrombosis and sepsis. Int. J Lab Hem., 00:1-8. https://doi.org/10.1111/ijlh.12800
Lefranc M. P. (1997). J. Immunol. Today, 18, 509.
Manda-Handzlik A., et al. (2016). Flow cytometric quantification of neutrophil extracellular traps: Limitations of the methodological approach. Am J. Hematol., 91. E9-E10.
Neeli I., Khan S. N., Radic M. (2008). Histone deimination as a response to inflammatory stimuli in neutrophils. J. Immunol. 180, pages 1895-1902.
Schneck E., et al. (2020). Flow Cytometry-Based Quantification of Neutrophil Extracellular Traps Shows an Association with Hypercoagulation in Septic Shock and Hypocoagulation in Postsurgical Systemic Inflammation—A Proof-of-Concept Study. J. Clin. Med., 9, 174.
Thompson J. D. et al., (1994). Nucleic Acid Res., 22, pages 4673-4680.
Van Dam L. S., Rabelink T. J., van Kooten C. and Teng Y. K. O. (2018). Clinical Implications of Excessive Neutrophil Extracellular Trap Formation in Renal Autoimmune Diseases. Kidney Int. Rep. 4(2), pages 196-211.
van der Linden M., Westerlaken G. H. A., van der Vlist M., van Montfrans J., Meyaard L. (2017). Differential signalling and kinetics of neutrophil extracellular trap release revealed by quantitative live imaging. Sci. Rep., 7, page 6529.
Verma R. et al., (1998). J. Immunol. Methods, 216, pages 165-181.
Wong I. and Lohman T. M. (1993). Proc. Natl. Acad. Sci. USA, 90, pages 5428-5432.
Zharkova O., et al. (2019). A Flow Cytometry-Based Assay for High-Throughput Detection and Quantification of Neutrophil Extracellular Traps in Mixed Cell Populations. Cytometry Part A, 95A, pages 268-278.
Zola H. (1987). Monoclonal Antibodies: a manual of techniques CRC Press

1. A method of identifying a human subject who will respond to administration of an agent that inhibits the formation of NETs, the method comprising:
(i) measuring the level of NETs and/or preNETs in a blood sample from said subject; and
(ii) identifying the subject as responsive to administration of the agent when the percentage of NETs to total number of neutrophils, or the percentage of preNETs to total number of neutrophils, in said sample is greater than 1%.

2. A method according to claim 1, further comprising administration of an agent that inhibits the formation of NETs to the human subject.

3. A method according to claim 2, further comprising measuring the level of NETs, and/or preNETs in a blood sample from said subject taken after administration of the agent.

4. A method of monitoring the effectiveness of a method of treatment in a human subject comprising:
(i) administration of an agent that inhibits the formation of NETs, or a binding fragment thereof, to the human subject;
(ii) measuring the level of NETs, and/or preNETs in a blood taken sample from said subject after administration of the agent.

5. A method according to claim 4, wherein before step (i) the level of NETs, and/or preNETs in a blood sample from said subject is measured.

6. A method of selecting a human subject for treatment with an agent that inhibits the formation of NETs, comprising:
(i) measuring the level of NETs and/or preNETs in a blood sample from said subject; and
(ii) identifying the human subject as responsive to administration of the agent when the percentage of NETs to total number of neutrophils, or the percentage of preNETs to total number of neutrophils, in said sample is greater than 1%; and
(iii) selecting the human subject for treatment with the agent based on the identification of the subject according to (ii).

7. A method according to claim 6, wherein the blood sample is a whole blood sample.

8. A method according to claim 6, wherein the blood sample is a peripheral whole blood cell sample.

9. A method according to claim 6, wherein the agent inhibits NET formation by preventing chromatin decondensation.

10. A method according to claim 9, wherein the agent is an antibody, or binding fragment thereof.

11. A method according to claim 10, wherein the antibody or binding fragment thereof, is a therapeutic anti-citrullinated protein antibody, or a binding fragment thereof.

12. A method according to claim 11, wherein the therapeutic anti-citrullinated protein antibody or binding fragment thereof is an antibody or binding fragment thereof that specifically binds to a citrullinated epitope on deiminated human histone 2A and/or histone 4.

13. A method according to claim 12, wherein the antibody or binding fragment thereof comprises the CDRs of SEQ ID NOs: 1, 2, 3, 9, 4 and 5.

14. A method according to claim 6, wherein measurement of the level of preNETs in the blood sample is carried out by FACS.

15. A method according to claim 6, wherein the human subject has a NET-associated pathology.

16. A method according to claim 15, wherein the NET-associated pathology is an inflammatory condition, an autoimmune condition or another NET-associated pathology such as wound healing in diabetes, cancer, cancer metastasis, transplant organ health in vivo or ex vivo, or viral induced acute respiratory distress syndrome (ARDS).

17. A method according to claim 16, wherein the condition is systemic lupus erythematosus (SLE), idiopathic inflammatory myopathy, lupus, sepsis, thrombosis, vasculitis, small-vessel vasculitis (SVV), antiphospholipid syndrome (APS), inflammatory arthritis, rheumatoid arthritis and osteoarthritis, psoriasis, Alzheimer's disease, autoimmune hepatitis, juvenile idiopathic arthritis, ulcerative colitis, Sjögren's disease, Anti-phospholipid Syndrome, Bechet's disease, spondylitis, spondyloarthropathy, multiple system atrophy, Parkinson's disease, Lewy body dementia, asthma, allergic rhinovirus exacerbated asthma, allergic asthma, cystic fibrosis, fibrosis and idiopathic pulmonary fibrosis, dry eye disease, uveitis, nongranulomatous uveitis, granulomatous uveitis, dermatitis, atopic dermatitis, inflammatory bowel disease, COPD or bronchitis.

18. A method according to claim 6, further comprising a cell fixation step.
